(19) United States
(12) Patent Application Publication
(10) Pub. No.: US 2025/0216488 A1
(43) Pub. Date: Jul. 3, 2025

(54) MAGNETIC RESONANCE APPARATUS, COMPUTER-ACCESSIBLE MEDIUM, SYSTEM AND METHOD FOR USE THEREOF

(71) Applicant: The Trustees of Columbia University in The City of New York, New York, NY (US)

(72) Inventors: John Thomas VAUGHAN, JR., New York, NY (US); Sairam GEETHANATH, New York, NY (US)

(21) Appl. No.: 18/779,934

(22) Filed: Jul. 22, 2024

Related U.S. Application Data
(63) Continuation of application No. PCT/US2023/011328, filed on Jan. 23, 2023.
(60) Provisional application No. 63/301,562, filed on Jan. 21, 2022.

(51) Int. Cl. G01R 33/56; A61B 5/055; G01R 33/36; G01R 33/38; G01R 33/3815; G01R 33/54; G06N 20/00; G06T 7/00

(52) U.S. Cl. CPC G01R 33/5608; A61B 5/055; G01R 33/3621; G01R 33/3804; G01R 33/3815; G01R 33/546; G06N 20/00; G06T 7/0012; G06T 2207/10088; G06T 2207/20081; G06T 2207/30016

(57) ABSTRACT

System, apparatus, method and computer-accessible medium according to exemplary embodiments of the present disclosure which facilitate accessible, data-driven neuroimaging. Exemplary embodiment of the present disclosure provides for a magnet, a gradient, and spectrometer technology that can be contained in an imaging suite that can be affordably manufactured, delivered, and operated anywhere in the world by a trained field nurse. Exemplary networks of these field-deployed MR suites can be tethered to a remote resource base such as a hospital through satellite links to a cloud platform. In exemplary embodiments, massive amounts of heterogeneous data can be collected from diverse populations in a standardized way and archived for machine learning approaches to permit model-based inference generation.

### CROSS-REFERENCE TO RELATED APPLICATION(S)

This application relates to and claims priority from U.S. Patent Application No. 63/301,562, filed Jan. 21, 2022, the entire disclosure of which is incorporated herein by reference.

### FIELD OF THE DISCLOSURE

The present disclosure relates generally to magnetic resonance systems, and more specifically, to exemplary embodiments of an exemplary resonance systems with cloud connectivity.

### BACKGROUND INFORMATION

Neuroimaging using MR technology may be limited to small cohorts of young adults studied in modern labs typically located in cities of affluent countries. As such, the rich tapestry of the human mind and brain may remain beyond our observation because our current MR measurement tools and methods cannot reach those living in other environments.

Thus, it may be beneficial to provide an exemplary magnetic resonance system which can overcome at least some of the deficiencies described herein above.

### SUMMARY OF EXEMPLARY EMBODIMENTS

To solve this problem and other problems, the present disclosure describes exemplary system, apparatus, method and computer-accessible medium for reinvention of MR technology, imaging methods, and data management. Indeed, one of the objects of the present disclosure is to provide systems, methods and computer-accessible medium which facilitate accessible, data-driven neuroimaging to transform human neuroscience and medicine on a global scale. For example, according to an exemplary embodiment of the present disclosure, it is possible to provide magnet, gradient, and spectrometer technology that can be contained in an imaging suite that can be affordably manufactured, delivered, and operated anywhere in the world by a trained field nurse. Exemplary networks of these field-deployed MR suites can be tethered to a remote resource base such as a hospital through satellite links to a cloud platform. In exemplary embodiments, massive amounts of heterogeneous data can be collected from diverse populations in a standardized way and archived for machine learning approaches to permit model-based inference generation. These exemplary next-generation MR can take safe and non-invasive, structural, metabolic, and functional neuroimaging to the world in the most literal sense, delivering a paradigm shift to science and medicine.

An exemplary object of the present disclosure is to describe a highly accessible MR system. Exemplary system, method and computer-accessible medium accordingly to an exemplary embodiment of the present disclosure can include:
Magnet: An exemplary 1-T, 80-cm bore magnet that can be sited and supported without dependence on modern infrastructure. This exemplary magnet and an exemplary associated MR system can be containerized in an “imaging suite” that can be affordably manufactured and delivered as a turnkey unit for use in any location or environment;
an exemplary B0 field control which can be effectuated via an exemplary innovative B0 coil hardware for spherical harmonic and multi-coil approaches that can be tailored to this magnet architecture for neuroimaging and state-of-the-art B0 field homogenization;
an exemplary new spectrometer that can be provided to maximize performance while minimizing cost and size;
exemplary console for real-time data acquisition “OCRA” can be provided which can be controlled by a Raspberry Pi (RPi) computer that can be connected to any smart device for local display; and
an exemplary software can be provided for data acquisition, image reconstruction, and user interaction that can be further developed and adapted to the new system hardware. Exemplary methods to reconstruct images acquired in inhomogeneous fields and with low RF power can also be utilized. Exemplary cloud-based autonomous MRI (AMRI) software can be developed to transfer MR data and protocols with minimal human intervention.

Another exemplary object of the present disclosure is to link MR systems with intelligent imaging in the cloud. Exemplary system, method and computer-accessible medium accordingly to an exemplary embodiment of the present disclosure can include, e.g., at least one of the following:
an exemplary cloud system which can be included or operate with a cloud-based network for the training of models, image analysis, and uploading and downloading of brain MRI data across multiple sites and vendors; and/or
an exemplary model-based imaging and inference which can utilize an exemplary that probabilistic model can capture the heterogeneity of normal human brain anatomy using representations based on deep neural network models as priors to improve images and inferences. The exemplary model can initially be trained on publicly available human MRI data and will provide priors that will improve both brain images (e.g., intelligent denoising and super resolution) and inferences (e.g., detecting deviations from normal anatomy).

Another exemplary object of the present disclosure can be to provide accessible MR imaging with a heterogeneous sample of human brains. Exemplary system, method and computer-accessible medium accordingly to an exemplary embodiment of the present disclosure can include, e.g., at least one of the following:
an exemplary data acquisition which can utilize exemplary structural MRI data that can be acquired from 120 healthy individuals spanning a wide range of ages (4 to 60 years, including preschool children and older adults) on the new accessible MR, a 1.5T, and a 3T system; and/or
an exemplary quantification of image quality which can utilize or work with an exemplary system that can be evaluated in terms of the quality of the images (before and after model-based enhancement) and resulting cortical-thickness maps. The 3T data can serve as a reference, enabling one to quantify image quality in terms of the mean squared error and structural similarity index measure (SSIM). Exemplary image quality between different variants of the new accessible system and the 1.5T can be compared inferentially, e.g., using the 3T as a reference in the same individuals.

Exemplary magnetic-resonance-based (MR) measurements, including structural and functional imaging as well as spectroscopy, are tools for safely and noninvasively observing anatomy, metabolism, and functional activation in the brain and body for science and medicine. According to the World Health Organization, two thirds of the world have no access to this powerful tool [see, e.g., Reference Nos. 2 and 3]. The reasons for limited or no access can be, e.g., geographic availability, cost, complexity, fragility, and dependence on modern infrastructure and expertise to operate MRI machines, prescribe data-acquisition protocols, and interpret their results.

An exemplary object of the present disclosure can be to provide and demonstrate a solution to this problem by bringing together new technologies, methods, and models to produce an MR system and the demonstrated means to make it accessible to all people, worldwide. Exemplary system, method and computer-accessible medium accordingly to an exemplary embodiment of the present disclosure can extend and distribute exemplary laboratories to the world's populations in their native environments to investigate the human mind and brain safely and noninvasively from birth to death will be a very significant achievement.

Exemplary system, method and computer-accessible medium accordingly to an exemplary embodiment of the present disclosure can include and/or integrate exemplary MR technologies and methods with a cloud-based machine learning and communications infrastructure at a global level.

Exemplary system, method and computer-accessible medium accordingly to an exemplary embodiment of the present disclosure can include:
(1) Exemplary MgB2 magnet that can be sited and operated off-the-grid, anywhere in the world.
(2) Exemplary coil hardware concepts for B₀-field control comprising spatial encoding (i.e., MRI) and B₀-field homogenization (i.e., shimming).
(3) Exemplary compact, efficient, high-performance, high-reliability spectrometer that can be housed entirely in the RF-coil package in the bore of the magnet.
(4) Exemplary data-acquisition controller and user interface (console) with a satellite link that is compact, efficient, reliable, and vendor-agnostic.
(5) Exemplary imaging systems and methods to maximize SNR, speed, efficiency, and reliability, while minimizing equipment and system complexity. Exemplary systems and/or methods can simultaneously transmit and receive (STAR), non-uniform field imaging, and imaging with hybrid fixed/dynamic gradients.
(6) Exemplary cloud-based autonomous data acquisition protocols, archiving, computing, and connection to resource centers.
(7) Exemplary machine-learning models for image denoising, super-resolution, and modeling of brain variability.
(8) Exemplary application of such exemplary models and accessible MRI to analyze diverse human brain structure, and to obtain greater diversity in large-scale developmental datasets acquired globally.

Exemplary systems, methods and computer-accessible medium accordingly to exemplary embodiments of the present disclosure can include a magnetic resonance (MR) system for diagnostic imaging including a magnet, a scanner, a radio frequency (“RF”) analog spectrometer, and an autonomous MRI software application configured to be activated through a mode of operation.

In exemplary systems, methods and computer-accessible medium accordingly to exemplary embodiments of the present disclosure, the magnet can be at least one of a superconducting, a solenoid, or a short solenoid with a nonuniform field of less than 5 ppm. In exemplary systems, methods and computer-accessible medium accordingly to exemplary embodiments of the present disclosure, the field nonuniformities of the magnet can be used for spatial encoding. In exemplary systems, methods and computer-accessible medium accordingly to exemplary embodiments of the present disclosure, a bore of the magnet can at least one of improve ergonomics, reduce claustrophobia, or reduce at least one of weight, size or cost of the magnet. In exemplary systems, methods and computer-accessible medium accordingly to exemplary embodiments of the present disclosure, the magnet can be made of at least one of HTSC or MgB2. In exemplary systems, methods and computer-accessible medium accordingly to exemplary embodiments of the present disclosure, the magnet can have a cooling system, which can have at least one of cryo-plate cooling, liquid H2 cooling, or solid N2 cooling, or does not have He2 cooling. In exemplary systems, methods and computer-accessible medium accordingly to exemplary embodiments of the present disclosure, the magnet can have at least one of an operating temperature of higher than 4.2K, relaxed manufacturing tolerances, or reduced cryostat.

Exemplary systems, methods and computer-accessible medium accordingly to exemplary embodiments of the present disclosure have at least one of a thermal reservoir to maintain a field for a time period without power or a local generator to energize the magnet. Exemplary systems, methods and computer-accessible medium accordingly to exemplary embodiments of the present disclosure can have at least one of a housed, operated or shielded in a half-sized or full-sized standard shipping container.

In exemplary systems, methods and computer-accessible medium accordingly to exemplary embodiments of the present disclosure, the spectrometer can have at least one of a an RF transmitter, an RF receiver, a TR switch, a circulator, an isolator, an analog to digital converter (“ADC”), a digital to analog converter (“DAC”), a single transmit signal channel, multiple transmit signal channels, a single receive channel, or multiple receive channels. In exemplary systems, methods and computer-accessible medium accordingly to exemplary embodiments of the present disclosure, the spectrometer can at least one of transmit and receive simultaneously, transmit and receive sequentially, or transmit simultaneously. In exemplary systems, methods and computer-accessible medium accordingly to exemplary embodiments of the present disclosure, the spectrometer can isolate transmit and receive by at least one of time, phase, frequency, space (geometry), or signal magnitude.

In exemplary systems, methods and computer-accessible medium accordingly to exemplary embodiments of the present disclosure, the spectrometer can at least one of transmit a magnitude modulated signal, transmit a phase modulated signal, transmit a time modulated signal, transmit a spatially modulated signal, transmit a frequency modulated signal, adjust receiver gain, adjust a receiver frequency and bandwidth, receive a signal modulated in time, receive a phase adjusted signal, or conduct spatial beam steering. In exemplary systems, methods and computer-accessible medium accordingly to exemplary embodiments of the present disclosure, the spectrometer can, using a broad-band receiver or transmitter, at least one of receive multiple nuclear resonance frequencies or excite multiple nuclear resonance frequencies.

In exemplary systems, methods and computer-accessible medium accordingly to exemplary embodiments of the present disclosure, the spectrometer can be controlled by a field programmable gate array (“FPGA”). Exemplary systems, methods and computer-accessible medium accordingly to exemplary embodiments of the present disclosure can further include at least one of a data acquisition unit, a digital user interface, a patient table, a field gradient system, a field shim set, an EMI shield, a magnetic fringe field shield, a secure enclosure, a support suite, or a radiofrequency coil.

Exemplary systems, methods and computer-accessible medium accordingly to exemplary embodiments of the present disclosure may not contain any rate earths. Exemplary systems, methods and computer-accessible medium accordingly to exemplary embodiments of the present disclosure can be networked with other MR systems using a cloud network. Exemplary systems, methods and computer-accessible medium accordingly to exemplary embodiments of the present disclosure can be networked using wireless or wired networking protocols. In exemplary systems, methods and computer-accessible medium accordingly to exemplary embodiments of the present disclosure, the other MR systems include scanners which are synchronized. In exemplary systems, methods and computer-accessible medium accordingly to exemplary embodiments of the present disclosure, the scanners can be configured to communicate using the cloud network to exchange at least one of protocols, data or predictive analysis.

Exemplary systems, methods and computer-accessible medium accordingly to exemplary embodiments of the present disclosure can be at least one of operationally sustainable, reliable, or deliverable using a transportation vehicle. In exemplary systems, methods and computer-accessible medium accordingly to exemplary embodiments of the present disclosure, the mode of operation is at least one of a voice command, a visual user-interface command, a QR code, a smart device. In exemplary systems, methods and computer-accessible medium accordingly to exemplary embodiments of the present disclosure, the mode of operation may not require human input to at least one of acquire, reconstruct, assess or report data.

In exemplary systems, methods and computer-accessible medium accordingly to exemplary embodiments of the present disclosure, the autonomous MRI software application can have an optimization image acquisition module for higher MR values; and the higher MR values can be diagnostic information per unit cost or unit time. In exemplary systems, methods and computer-accessible medium accordingly to exemplary embodiments of the present disclosure, the optimization image acquisition module can interact with a scanner on a cloud. In exemplary systems, methods and computer-accessible medium accordingly to exemplary embodiments of the present disclosure, the autonomous MRI software application can determine acquisition parameters through at least one of integration of MR physics, AI search strategies, patient derived statistics, or electronic health records. In exemplary systems, methods and computer-accessible medium accordingly to exemplary embodiments of the present disclosure, the autonomous MRI software application is configured to denoise MR data to accelerate acquisitions using at least one of native or learned noise structures. In exemplary systems, methods and computer-accessible medium accordingly to exemplary embodiments of the present disclosure, the autonomous MRI software application can utilized transfer learning to leverage native noise denoising.

In exemplary systems, methods and computer-accessible medium accordingly to exemplary embodiments of the present disclosure, the autonomous MRI software application can be configured to integrate at least one of cognizance, reflectivity, adaptivity or ethical compliance rules to transform the MR system into an intelligent system. In exemplary systems, methods and computer-accessible medium accordingly to exemplary embodiments of the present disclosure, the autonomous MRI software application can incorporate cognizance through intelligent slice planning. In exemplary systems, methods and computer-accessible medium accordingly to exemplary embodiments of the present disclosure, the autonomous MRI software application is configured to incorporate at least one of cognizance through intelligent slice planning, reflectivity through intelligent protocolling, adaptivity through user intervention for MR exams, taskability through voice interaction, or ethical behavior through patient information encryption in speech to text or text to speech transformations.

According to exemplary embodiments of the systems, methods and computer-accessible medium of the present disclosure, the autonomous MRI software application can optimize for increased value a ratio of diagnostic information to a cost, wherein at least one of the diagnostic information is related to qualitative MR contrasts or quantitative tissue parametric maps; and the cost is associated with a time spent in the scanner or scanning fees. In exemplary systems, methods and computer-accessible medium accordingly to exemplary embodiments of the present disclosure, the autonomous MRI software application is configured to enable a remote operation through at least one of self-scanning, monitoring, performing consistency checks, flagging degradation or escalating potential failure modes.

In exemplary systems, methods and computer-accessible medium accordingly to exemplary embodiments of the present disclosure, at least one of the self-scanning is accomplished by the interplay between a user-node, a cloud and the scanner with a user-node controlling the other two components; or the monitoring of the scanner is performed by the use of acquisition associated with a pattern recognition technique. Further, in exemplary systems, methods and computer-accessible medium accordingly to exemplary embodiments of the present disclosure, the autonomous MRI software application can utilize pattern recognition outputs of an acquisition to classify patterns associated with a system status and a degradation status. In exemplary systems, methods and computer-accessible medium accordingly to exemplary embodiments of the present disclosure, the autonomous MRI software application can flag at least one of system degradation of hardware and networking components including at least one of the magnet, a gradient, or a cloud connectivity. In exemplary systems, methods and computer-accessible medium accordingly to exemplary embodiments of the present disclosure, the autonomous MRI software application can control a console comprising a field programmable gate array (“FPGA”) device. In exemplary systems, methods and computer-accessible medium accordingly to exemplary embodiments of the present disclosure, the FPGA device can adhere to standards for pulse sequence programming; the FPGA device includes a large range of transmit and receive channels; the FPGA device can operate at high sampling rates to accommodate high speed streaming of data experienced in simultaneous transmit and receive acquisitions; or the FPGA device can provide real-time feedback to a user-node to correct for artifacts including patient motion or load changes.

According to additional exemplary embodiments of the exemplary systems, methods and computer-accessible medium of the present disclosure the autonomous MRI software application is configured to interface with an image guided radiation therapy platform.

In exemplary systems, methods and computer-accessible medium accordingly to exemplary embodiments of the present disclosure, the scanner can at least one of acquire images in inhomogeneous fields; integrate electromagnetic simulation and pattern recognition-based acquisition; capture image in highly non-uniform magnetic fields to account for a short bore length; utilize pattern recognition methods including at least one of fingerprinting, frequency swept pulses, or selective excitation; or encode one whole image in a single echo with multiple receiver coils.

According to additional exemplary embodiments of the exemplary systems, methods and computer-accessible medium of the present disclosure, the single echo at least one of can achieve acquisition times of an order of the echo times of the desired contrast; reduce radio-frequency power deposited in a patient compared to gold-standard spin and gradient echo sequences; reduce peripheral nerve stimulation in patients compared to gold-standard spin and gradient echo sequences; reduce gradient noise compared to gold-standard spin and gradient echo sequences.

In exemplary systems, methods and computer-accessible medium accordingly to exemplary embodiments of the present disclosure, the scanner can at least one of generate common contrasts including T1 weighted, T2 weighted, or diffusion weighted imaging, using conventional and simultaneous transmit and receive methods; utilize pattern recognition acquisition-reconstruction methods to produce quantitative tissue parametric maps to simultaneously generate qualitative and quantitative MR data; utilize a vendor-neutral, open source library for development to aid rapid prototyping and development; generate acquisitions in a web-browser to enable cloud generation of acquisition files; utilize pattern recognition methods to generate tissue specific magnetization evolutions to provide quantitative imaging parameters including T1-map, T2-map, or apparent diffusion coefficient map; gauge and detect system degradation including the deterioration of the coils, or console, using pattern recognition methods; estimate temperature using new pulse sequences to provide safety checks above and beyond specific absorption rate methods.

In exemplary systems, methods and computer-accessible medium accordingly to exemplary embodiments of the present disclosure, the scanner can at least one of acquire images in inhomogeneous fields using deep learning; exploit system priors including B0 or B1 fields, or subject priors including anthropomorphic details, or cardiac motion pattern, to integrate intelligence in image reconstruction. In exemplary systems, methods and computer-accessible medium accordingly to exemplary embodiments of the present disclosure, the deep learning can at least one of obtain accurate and robust reconstruction in the presence of noise and motion, speed up acquisition or provide repeatable quantitative imaging measures.

In exemplary systems, methods and computer-accessible medium accordingly to exemplary embodiments of the present disclosure, the deep learning can at least one of reconstruct data from Cartesian and non-Cartesian trajectories to accelerate image reconstruction computation and reduce artifacts due to aliasing or gridding; translate pattern recognition derived acquisitions to compute quantitative maps in an accelerated manner; or utilize cloud or local computing to perform reconstruction methods related to Cartesian or non-Cartesian data.

In exemplary systems, methods and computer-accessible medium accordingly to exemplary embodiments of the present disclosure, the reconstruction methods can include at least one of conforming to global (file) standards on acquisition, reconstruction, image analysis or communication (DICOM); or transformation of raw data to clinically valuable and interpretable quantitative parametric maps or statistics. In exemplary systems, methods and computer-accessible medium accordingly to exemplary embodiments of the present disclosure, the maps can facilitate clinical assessment or enable inclusion of Electronic Health Record obtained and MR data to predict trends and outcomes. In exemplary systems, methods and computer-accessible medium accordingly to exemplary embodiments of the present disclosure, the reconstruction methods can estimate quantitative MR parameters jointly through randomization of acquisition parameters, estimating gradient warp and non-linearities through calibration and deep learning, or motion estimation through signal analysis from the gradient and radiofrequency coils.

In exemplary systems, methods and computer-accessible medium accordingly to exemplary embodiments of the present disclosure, the autonomous MRI software application can include a quality assurance module configured to at least one of guarantee standardization of image quality by flagging presence of artifacts including wrap around, Gibbs ringing, or motion artifacts, during scan time to enable rescans; check for consistent scanner operation, consistent coil performance, anatomy coverage, or missing acquisitions in protocol to provide a baseline image quality for downstream analysis; calculate image quality metrics including reference and non-reference methods to track image quality over time to detect any potential scanner degradation; or identify, recognize and report system degradation based on predetermined responses to random configurations of test signals on each of the hardware components.

In exemplary systems, methods and computer-accessible medium accordingly to exemplary embodiments of the present disclosure, the quality assurance module can include random gradient waveforms to test pre-determined point spread functions of such a k-space trajectory. In exemplary systems, methods and computer-accessible medium accordingly to exemplary embodiments of the present disclosure, the scanner can run multiple diagnostic applications related to different anatomies and pathologies. In exemplary systems, methods and computer-accessible medium accordingly to exemplary embodiments of the present disclosure, the autonomous MRI software application can at least one of translate MR data and images into clinically meaningful metrics to characterize structure, function or metabolism of an anatomy of interest; utilize deep learning to calibrate quantitative imaging outcomes per subject and per population; generate a subject-readable report using deep learning that combines subject information, imaging data or radiologist's expertise; provide a digital health record that evolves over time to record a transition of health to disease and potential reversal; or be accessed via an application store on a smart device by users in a configurable manner.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is an illustration of an exemplary MR system according to exemplary embodiments of the present disclosure;

FIG. 2 is an illustration of an exemplary magnesium diboride (MgB2) magnet accordingly to an exemplary embodiment of the present disclosure;

FIG. 3 is a set of images and a graph providing exemplary preliminary results of REMODEL for off-resonance frequencies up to ±30 kHz with a 4 shot Echo Planar Imaging k-space trajectory;

FIGS. 4a-4c are a set of graphs providing exemplary results for an exemplary region of an exemplary interest analysis according to an exemplary embodiment of the present disclosure;

FIG. 5 is a set of illustration of an exemplary magnet accordingly to an exemplary embodiment of the present disclosure;

FIG. 6a is an exemplary graph of shows exemplary signal intensities of three brain matters as a function of flip rates;

FIG. 6b is a set of exemplary illustrations of three exemplary contrasts of the brainweb numerical phantom with different flip rates;

FIG. 7 is an exemplary end-to-end Pulseq-GPI workflow according to an exemplary embodiment of the present disclosure;

FIG. 8 is an exemplary follow diagram of cloud networking procedure according to an exemplary embodiment of the present disclosure;

FIG. 9 is an exemplary graph of exemplary results generated according to an exemplary embodiment of the present disclosure;

FIG. 10 is a set of illustration of exemplary results on improving low-quality images with a reverse model implemented in a deep neural network trained with supervision according to an exemplary embodiment of the present disclosure;

FIG. 11 is a flow diagram of an exemplary neural network probabilistic model of human brain anatomy according to an exemplary embodiment of the present disclosure; and

FIG. 12 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

### Exemplary MR System

Exemplary system, method and computer-accessible medium accordingly to an exemplary embodiment of the present disclosure can include, e.g., a magnet. For example, exemplary systems accordingly to the present disclosure can relieve some of the constraints that the magnet places on accessibility. To distinguish between other so-called “portable” magnets, the present disclosure may prioritize, e.g., access and performance over the ability to move a magnet from site to site, though it is moderately portable as well. The magnets currently being marketed as portable are either laboratory-bound and dependent on significant infrastructure and expertise to operate, or of ultra-low field strengths limiting their performance for many/most neuroscience applications envisioned. By contrast, the present disclosure can prioritize a magnet of “clinical, diagnostic quality” field strength that can be packaged, delivered and operated anywhere with minimum infrastructure, expertise or field support. This magnet may need to be reliable, helium free, and cost effective too.

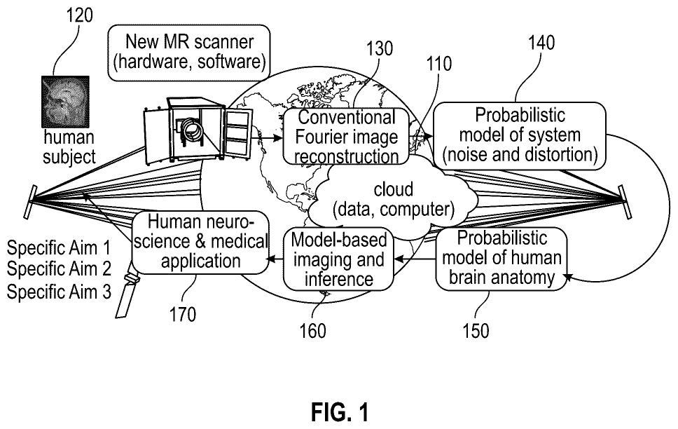

FIG. 1 shows an exemplary MR system according to exemplary embodiments of the present disclosure. As shown in FIG. 1, exemplary system, method and computer-accessible medium accordingly to an exemplary embodiment of the present disclosure can include, e.g., a network of remotely accessible MR systems with satellite links to a cloud platform 110. Managed from a remote hospital or laboratory, image-acquisition protocols can be downloaded, and acquired brain image data 120 can be uploaded to, archived, and curated in the cloud 110. Exemplary embodiments of the present disclosure can facilitate a generation of a harmonized collection of neurological brain data of unprecedented dimension and diversity. Exemplary embodiments of the present disclosure can be a single, scalable, cloud-satellite linked MR system. Exemplary embodiments of the present disclosure can further process the brain image data 120 using conventions Fourier image reconstruction procedure 130, probabilistic model system 140, probabilistic model of human brain anatomy 150, model-based imaging and inference 160, and human neuro-science and medical application 170.

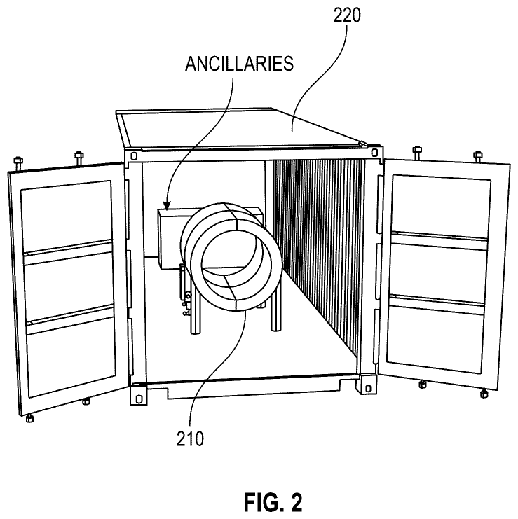

FIG. 2 shows an illustration of an exemplary magnesium diboride (MgB2) magnet 210 accordingly to an exemplary embodiment of the present disclosure. The exemplary magnesium diboride (MgB2) magnet of the form-factor shown in FIG. 2 can be delivered and sited in its own secure, weather-proof, half-sized shipping container 220 which can include all other components of the exemplary accessible MR system as well. This exemplary self-contained “MR suite” can replace the current three-roomed clinical installation consisting of a magnet room, equipment room and console room. It can be delivered in numerous ways, e.g., by ship, rail, plane, helicopter, or truck, placed on the ground or floor and operated turnkey with minimal site preparation or engineering. The container can double as a magnet and electromagnetic (EM) shield.

The exemplary MgB2 magnet can be operated in persistent mode at liquid hydrogen temperature of 20.28 K or solid nitrogen below 63K. Both of these cryogens can be extracted cheaply from water or air, unlike liquid helium (4.2K). And a magnet operating at higher temperatures can be more stable and tolerant of manufacturing imperfections, and can be more robust in surviving harsh delivery and extreme field siting conditions. Similarly, this exemplary magnet can be cooled with cryoplates as a plug-in option. This can be the configuration of this exemplary magnet.

Compared to a conventional Niobium Titanium, helium cooled magnet, the HTSC magnet may require less cryostat size, complexity and cost. This exemplary MgB2 magnet can be manufactured with modest economy of scale, and thus, can be cost competitive with current NiTi magnets of same bore dimensions and field strength. In an effort to conserve size, weight and cost of 800 mm bore magnet, the field homogeneity constraint may be relaxed for body imaging while the homogeneity over a shimmed 20 cm diametrical spherical volume (DSV) may remain well within the ability to correct. (See, e.g., Reference No. 69). The exemplary magnets' gradient and shim coils can be custom designed head gradient set package from Tesla Engineering Ltd, UK. The details are in Table 2.

TABLE 1
Exemplary Magnet Specifications

| Parameter | Reference Value |
|---|---|
| Main field | 1.0 T |
| Homogeneity over 20 cm DSV | 35 ppm |
| (passively shimmed) | |
| Magnet length | 1250 mm |
| Magnet height | 1450 mm |
| Magnetic center | 950 mm from the floor |
| Magnet mass | 800 kg |
| Transport | Room temperature |

TABLE 2
Exemplary Gradient Coils and Shims

| Parameter | Reference Value |
|---|---|
| Dimensions-Internal/external | 620 mm/770 mm |
| diameter | |
| Strength per amp | X/Y: 59.5 uT/m/A; Z: 59.2 uT/m/A |
| Residual eddy currents | <1% |
| Peak strength | 53.6 mT/m |
| Slew Rate | X: 227 T/m/s; Y: 211 T/m/s; Z: 264 T/m/s |
| Passive shim arrays | 24 trays (with 24 or 15 pockets) |
| Resistive shims-peak current | 6 shims ( [symbol] and 5 second order) @ 5 A |
| | peak current |
| Cooling system | Water cooled |

[symbol]

indicates data missing or illegible when filed

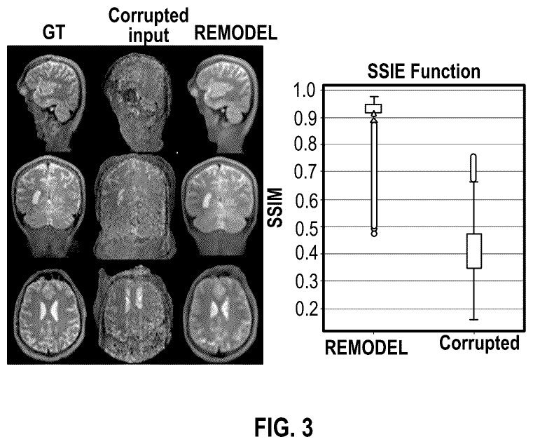

Magnetic field inhomogeneity: The short length magnet may cause inhomogeneity, which can be mitigated by an exemplary model based reconstruction that can be hardware cognizant. This can include incorporating measured field maps and building forward models that corrupt acquired data due to off-resonance. In particular, exemplary preliminary results of retrospectively reconstructing data corrupted with an off-resonance frequency range of up to ±30 kHz with a four shot EPI can be built. FIG. 3 shows exemplary intensity profile set and a graph of exemplary results of reconstruction of MR images in highly inhomogeneous fields using deep learning for off-resonance frequencies up to ±30 kHz with a 4 shot Echo Planar Imaging k-space trajectory. In this Figure, GT stands for Ground Truth and SSIE stands for Structural Similarity Index Estimate. (See, e.g., Reference No. 69). This exemplary procedure can utilize a three layer deep U-net followed by a final convolutional layer. This exemplary procedure can handle ˜20 ppm inhomogeneity expected in a 20 cm DSV for the exemplary magnet. This exemplary model can be further refined by training it on 20,000 brain volumes from the UK biobank (See, e.g., Reference No. 6) for T₁ and T₂ contrasts, and augmenting by alternative neural network architectures (See, e.g., Reference No. 7).

Spatial Encoding: Exemplary system, method and computer-accessible medium accordingly to an exemplary embodiment of the present disclosure can utilize the potential of replacing the phase encoding gradient with phase arrays. This can, e.g., increase MR efficiency by significantly reducing acquisition time by a factor of N_{PE} (number of Phase Encodes) with respect to conventional 2 dimensional Cartesian imaging and provide robust motion insensitivity due to the acquisition time of an entire image being lesser than Repetition Time (TR). The power requirements of the one of the three Gradient Power Amplifiers can be reduced to 0 W.

In addition, exemplary phase arrays can, e.g., reduce the gradient noise from the phase encoding gradient to 0 dB. (See, e.g., Reference No. 8). The exemplary phase arrays can incorporate spatially varying point spread functions, dubbed “replacing Phase Encoding gradients with Phased Arrays” (PEPA). PEPA's practical feasibility can be demonstrated using a 64 channel head and neck coil on a two water bottle phantom. The source code for reproducing results on numerical phantoms (with complex noise) and prospective in vitro experiments can be found are available. (See, e.g., https://github.com/imr-framework/PEPA).

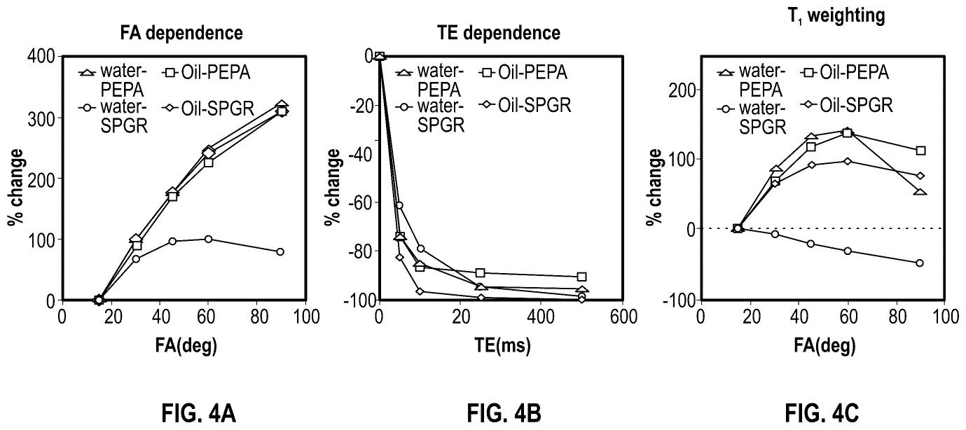

FIGS. 4a-4c show a set of graphs which provide an exemplary region of Interest analysis. For example, in FIG. 4a, FA dependence of signal intensities of water and oil for PEPA and control Spoiled Gradient Recalled Echo sequences, with a matrix size of 64×64, are shown. In FIG. 4b, TE dependence is illustrated. In FIG. 4c, T1 weighting is shown.

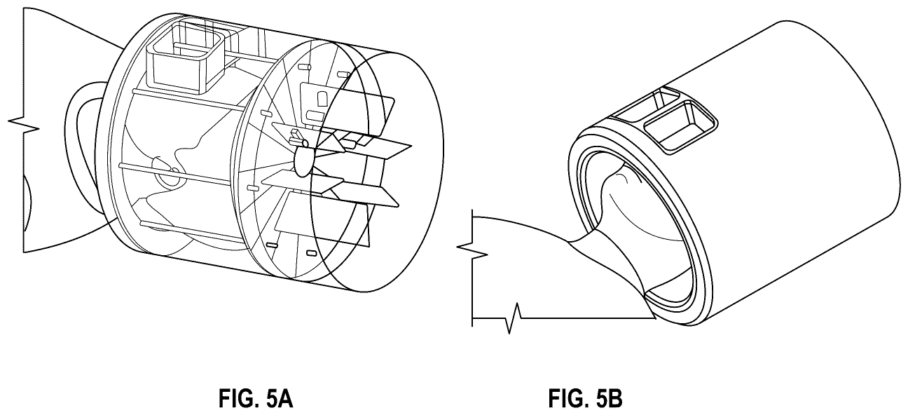

Further, an exemplary spectrometer for this exemplary system can include the radiofrequency (RF) head coil, the transmit-receive switches or circulators, the GaAsFET preamplifiers and the distributed powerFET power amplifiers. At this exemplary frequency of 42 MHz, the exemplary system can sample the signal directly with 16 bit ADCs immediately following the preamps. The RF coil can include of a circularly polarized, TEM transceiver coil. FIG. 5 shows an exemplary magnet accordingly to an exemplary embodiment of the present disclosure.

In exemplary embodiments of the present disclosure, two or more plans can be provided and tested for this RF spectrometer. For example, the first plan can be a more conventional and proven approach using eight, 2 kW pulsed RF power amplifiers located at the back of the magnet to drive the eight-element coil in parallel transmit fashion. Transmit receive switches can isolate the transmit pulse from received FID signals in time. The received signals can be detected and processed by conventional methods. The second plan can be the exemplary beneficial plan that can also be pursued. In this beneficial plan according to the exemplary embodiments of the present disclosure, exemplary 10W power FETS soldered to the coil elements for heat sinks, can drive the RF coil transmit per element. These FETS can be notch filter and phase isolated from the receivers. The exemplary coil can be driven with a CW, RF signal set to maximize (e.g., 90°) a continuous receive signal, and simultaneously transmit and receive (STAR, Sohn MRM 2016). This exemplary approach may cost conventional T₁ and T₂ decay curve contrast, but can maximize SNR. It can also reduce the entire RF/analog spectrometer of a system to a size and weight that, e.g., conveniently fits into the head coil package.

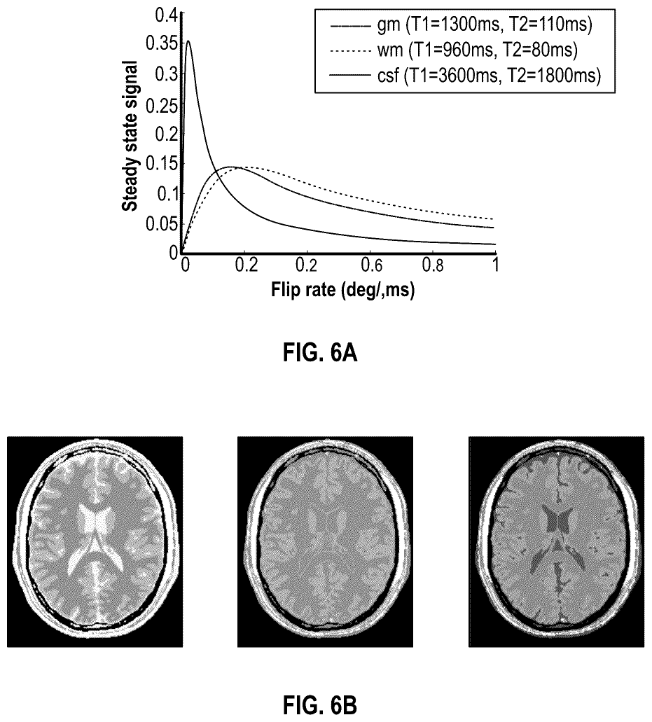

Exemplary STAR contrast: Exemplary system, method and computer-accessible medium accordingly to an exemplary embodiment of the present disclosure can employ tailored variations in flip rates (deg/ms) to reach different steady state magnetization patterns, delivering multiple contrasts. FIG. 6 (a) shows a graph of exemplary signal intensities of three brain matters as a function of flip rates. FIG. 6 (b) shows three exemplary contrasts of the brainweb numerical phantom with different flip rates. (See, e.g., Reference No. 17). Exemplary system, method and computer-accessible medium accordingly to an exemplary embodiment of the present disclosure can build on these simulations to deliver pulse sequences that offer T1, T2 and PD contrasts. In particular, the exemplary system can leverage the Extended Phase Graph (EPG) look ahead algorithm (See, e.g., Reference No. 12) to tailor the flip rates to yield desired contrasts at the earliest possible steady state. To accelerate acquisition, the exemplary system can leverage methods based on compressed sensing using a radial trajectory (See, e.g., Reference No. 13) and/or PEPA acquisitions.

Exemplary system, method and computer-accessible medium accordingly to an exemplary embodiment of the present disclosure can utilize and/or be based on the Open Source Console for Real-time Acquisition (OCRA) built on the Red-Pitaya (RP) board. (See, e.g., Reference No. 14). The exemplary system can make three specific modifications to this console architecture: (i) modify the client and FPGA software to make it compatible with pypulseq (See, e.g., Reference No. 70); (ii) extend the FPGA capability (either through an add-on to RP or a later generation board) to interface with increased number of I/O ports through a digital I/O board with 8 RF transmit and up to 64 receive channels. All required pulsing (gradients, shims) can be performed at 100 ns resolution, RF with a phase resolution of 0.0050 using a Direct Digital Synthesizer, and up to 12.5 MHz digital receiver bandwidth. (iii) explore new generations of FPGA boards that can integrate (i) and (ii) on a single board or a minimum number of off-the shelf boards. The utility of modifications (i) and (ii) can facilitate benchmarking with existing open source architecture and extending it to open source pulse sequence development (see, e.g., Reference Nos. 70 and 71) as well as build on an architecture that can leverage “off-the-shelf” firmware components that are easy to assemble, program and control with a high degree of safety. To this end, exemplary OCRA platform can be setup by following the instructions at https://openmri.github.io/ocra/hardware in collaboration with the CMRRC advanced instrumentation platform.

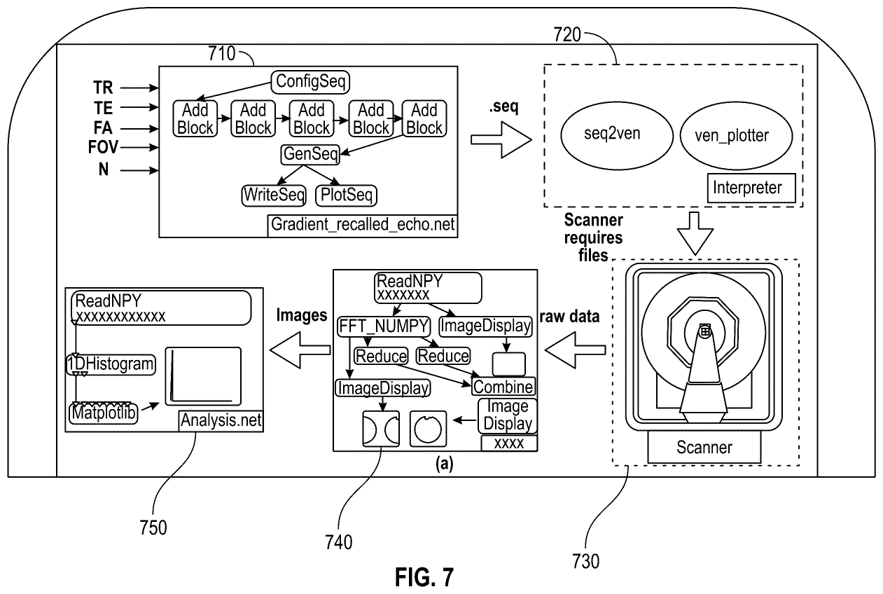

FIG. 7 shows an exemplary flow diagram providing an exemplary end-to-end Pulseq-GPI workflow. In this exemplary system and method, the console can facilitate a direct control of the scanner hardware without the need for a vendor interpreter. These vendor specific interpreters can be utilized for the GE and Siemens scanners.

Many of all of the exemplary methods (including the exemplary methods described herein) can include an exemplary system software which can leverage standard file formats for interfacing between the four SA deliverables. In particular, these standards are annotated in FIG. 1 in darker font. The use of such vendor-neutral standards (Pulseq, for example) is a design goal to enable scalability to similar prototypes and/or other existing MR systems. The system Graphical User Interface (GUI) can leverage “Virtual Scanner” interface (See, e.g., Reference No. 11) which requires only a web browser to run. This can be implemented in Python and host a central server tending simultaneously to multiple web client requests. The tool can be capable of generating Pulseq files (.seq) for pulse sequences, as shown in procedures 710 and 720 of FIG. 7. Therefore, the web GUI can be interfaced to the console via pypulseq (e.g., as shown in procedures 740 and 750 of FIG. 7). (See, e.g., Reference No. 70). The MR technician can run this exemplary tool in standard mode (e.g., as shown in procedure 730 of FIG. 7) while the advanced mode can be used by researchers and developers. The exemplary tool can leverage the Pulseq-Graphical Programming Interface (GPI) library (see, e.g., Reference No. 70) for comprehensive MR method development, either in GUI mode or in scripting mode. The pulseq-GPI library was developed by translating MATLAB Pulseq code into Python and integrating with GPI. The exemplary system can follow the Agile methodology of software development and continuous integration practices.

### Exemplary Cloud System

Exemplary system, method and computer-accessible medium accordingly to an exemplary embodiment of the present disclosure can comprise a cloud based storage network infrastructure and distributed machine, deep learning environment that can be used to harness the wealth of big data of acquired brain scans into the future. An exemplary universal interface between multiple vendors and sites is provided, which can leverage existing network infrastructure of the cloud for both computation, acquisition, analysis, storage and archive.

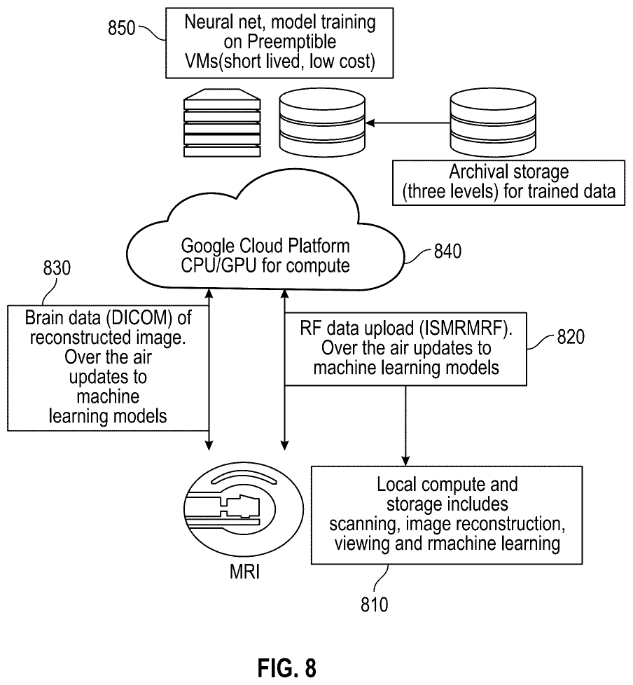

FIG. 8 shows an exemplary diagram of cloud networking according to an exemplary embodiment of the present disclosure. This exemplary networking supports preemptive computing, which can include three levels of hierarchical storage for training and upload, downloading via satellite connectivity and uplinks.

Exemplary Connectivity Uploading/downloading of data can be accomplished through two phases of connectivity: (1) Internal Development—Connections for local network, hub, subnets, etc., e.g., connections to local compute 810 of FIG. 8 (2) Deploy—Utilize satellite uplink/downlink connections for remote site testing, e.g., as shown in connections 820 and 830 of FIG. 8. At remote sites, each MRI machine shall be connected via a small local area network hub connected to a satellite dish (transmitter and receiver). Further, exemplary acquisition and image analysis can be implemented using exemplary system, method and computer-accessible medium accordingly to an exemplary embodiment of the present disclosure, which can acquire T1-MPRAGE, and T2-TSE images of 120 healthy volunteers across three age groups. The T1 and T2 sequences have been tested for contrast and SNR as part of the Autonomous MRI (AMRI) package (See, e.g., Reference No. 70). The exemplary system can adopt and/or utilize this cloud based AMRI acquisition-reconstruction software 850 to deliver required MR investigations with minimal human intervention and aid less experienced MR technicians (See, e.g., Reference No. 70). The AMRI package can interact with three components: (i) an exemplary user node-any smart device such as a laptop for user input and image visualization; (ii) the exemplary cloud 840 for intelligent protocolling, slice planning, compute and data storage; and (iii) an exemplary scanner for data acquisition. AMRI, therefore, can transform a standard MR system into an intelligent physical system (See, e.g., Reference No. 70). The exemplary system can integrate AMRI's user node software with the virtual scanner package (SA 1.D) and leverage GCP for the cloud functions. The exemplary system can leverage the open source, vendor-neutral accelerated pulseq based MR Fingerprinting package (See, e.g., Reference No. 32) to reduce the scan time for T1 and T2 acquisitions for pediatric populations, in case of increased motion artifacts.

Exemplary Image quality control: The MRI parameter and measurement evaluation can be conducted immediately after acquisition of each MRI scan and can be used as feedback for algorithm and parameter adjustment for MRI acquisition and MRI post-processing.

The accuracy and precision of exemplary systems, methods and computer-accessible medium according to exemplary embodiments of the present disclosure can be evaluated in two stages: 1) remodel of corrupted input and existing ground truth scans (n=300); 2) scans of healthy volunteers (n=120) acquired on AMRI and standard MRI systems (1.5T and 3T). The parameters that can be compared include root mean square error, structural similarity index, peak signal to noise ratio, contrast to noise ratio etc. Biomarkers such as cortical thickness and prefrontal cortex shape measurements can also be evaluated. The power calculation was conducted for a non-inferiority study design. If there is truly no difference between evaluated scans and ground truth scans, then 300 patients are required to be 80% sure that the lower limit of a one-sided 95% confidence interval (or equivalently a 90% two-sided confidence interval) will be above the non-inferiority limit of −0.29 of SD of an MRI parameter or measure. Similarly, then 120 patients are required to be 80% sure that the lower limit of a one-sided 95% confidence interval (or equivalently a 90% two-sided confidence interval) will be above the non-inferiority limit of −0.46 of SD of an MRI parameter or measure.

Exemplary regression models can be constructed when beneficial or necessary. Potential confounders such as vendors (i.e., Siemens and GE) can be coded as categorized variables and can be tested for significance in the regression models. Once the sequence has been acquired, both DICOM and ISMRMRD (raw RF data) can be stored. Below are exemplary detailed accounting of the data flow and bandwidth requirements to collect/gather MRI brain data from local site and implement ML/DL training and storage on the cloud. Compute and storage shall be at the device level (reconstruction can be performed in the cloud in the early stages while acquiring data.

Exemplary Archiving: As shown in FIG. 8, certain cloud platform (e.g., Google, etc.) can provide, e.g., multiple (e.g., three) levels of archiving service for training data, with a decreasing cost model: (a) “hot” storage for immediate access, (b) “warm” storage for data that should remain available during a current/ongoing analysis and (c) “cold” storage for long term archive modes. The data lake created in this project will be available for the neuroscience and MR communities to download and use for research purposes. The logistics of this archival process will be setup during year 4.

Exemplary Model-based imaging and inference. Exemplary system, method and computer-accessible medium accordingly to an exemplary embodiment of the present disclosure can leverage the large and heterogeneous data sets from the cloud to create machine learning models for image enhancement and specific inferences relevant to human neuroscience and medicine. Exemplary systems can use probabilistic representations based on deep neural network (NN) models for model-based improvement of MRI of human brain anatomy. The exemplary models can learn the statistical structure of the signal (i.e., the distribution of human brain images) and the noise (including instrumental noise, signal inhomogeneity, and physiological variation). Once learned, the exemplary models can provide priors that will improve both images and inferences based on accessible MRI. The exemplary models can equally be applicable to MRI at 3T or 7T, and can impact the use of such systems as well. However, the models can be a component of accessible MRI, which, on the one hand, may require stronger prior information for high-quality images and inferences and, on the other hand, can provide unprecedented amounts of data through the cloud for refining the required priors.

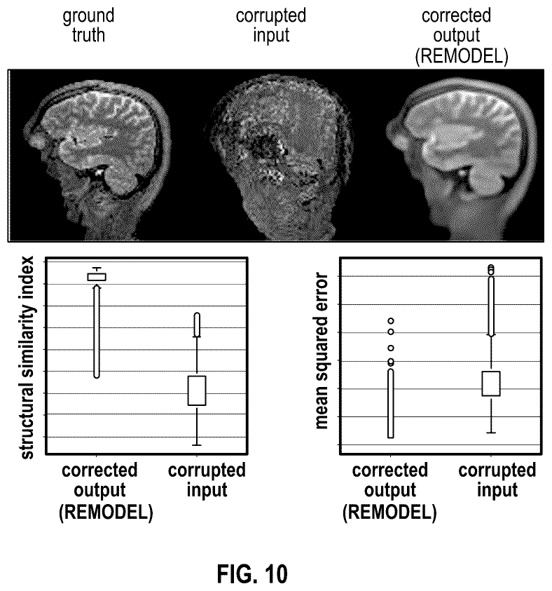

Exemplary Forward model of MR images. Exemplary system, method and computer-accessible medium accordingly to an exemplary embodiment of the present disclosure can leverage and extend the Virtual Scanner software described above to create a forward model (FM; See, e.g., Reference No. 17; xc→y; gray in FIG. 10) that simulates the MR images and tissue parametric maps that can be produced by the exemplary accessible MRI system.

FIG. 10 shows exemplary preliminary results on improving low-quality images with a reverse model implemented in a deep neural network trained with supervision [see, e.g., Reference No. 5]. The FM can simulate subsampling, noise, and subject- and anatomy-specific artifacts, including motion, distortion, and susceptibility artifacts. The FM can also involve mapping quantitative tissue parameters (T1, T2) to enable comparisons with control images from 1.5T and 3T systems. Exemplary system, method and computer-accessible medium accordingly to an exemplary embodiment of the present disclosure can use 3T T1-weighted anatomical images from a large number of humans from available databases, including the UK Biobank ([See, e.g., Reference No. 6]>40 K datasets). Exemplary systems can use data augmentation to further increase the number of ground-truth images available to the FM. The exemplary data augmentation can include linear operations of rotation, scaling, and translation, along with global smooth nonlinear distortions of the 3D volumes, based on a realistic model of the natural distribution of distances between brain anatomical landmarks (the anterior and posterior commissures, the anterior and posterior, and rightmost and leftmost points as used to define Talairach space). For a given ground-truth MRI volume xc in canonical position and orientation, the FM can first simulate a random translation and rotation T (xc), resulting in a randomly placed high-quality volume x. The FM can then simulate the lower-quality images y that we expect to obtain with the new hardware and image reconstruction.

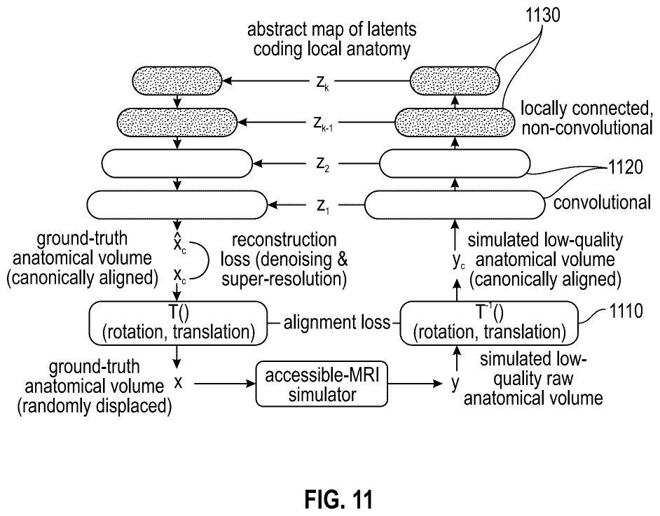

Exemplary Reverse model for denoising and super-resolution. Exemplary system, method and computer-accessible medium accordingly to an exemplary embodiment of the present disclosure can use the forward model (FM, xc→y, gray in FIG. 11) to generate a large number of training pairs of high-resolution, low-noise images xc and simulated low-resolution, high-noise images y (as expected from accessible MRI).

FIG. 11 shows a flow diagram of an exemplary neural network probabilistic model of human brain anatomy according to an exemplary embodiment of the present disclosure. The exemplary model can be trained on existing data in Phase 1 of training. It can be continually improved based on data flowing in through the cloud from the exemplary system during Phase 2 of training (via simulated remote sites) and deployment. The model can serve as a prior that can improve imaging and inferences, providing denoising and super resolution. Forward model (gray), reverse model (green, blue, red).

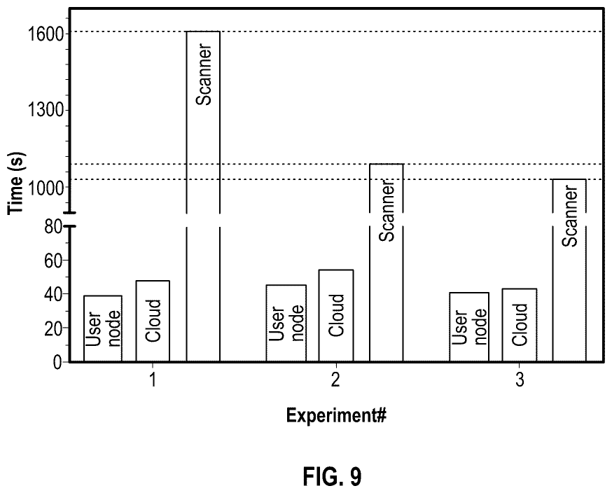

Exemplary system, method and computer-accessible medium accordingly to an exemplary embodiment of the present disclosure can train a reverse model (RM, y→∧xc) that takes accessible MRI images y as input and outputs an estimate ∧xc of the corresponding high-resolution, low-noise image xc. The exemplary trained model can denoise the data from the new accessible MRI system, correct for field inhomogeneities, and increase the image resolution. Because the FM, like any simulation, may not be perfect, the RM can require, in one embodiment, tuning and reconfiguring based on actual data acquired with the new accessible MRI system. However, the exemplary FM can enable an initial RM to be trained and tested to bootstrap the learning process. (See, e.g., Reference No. [see, e.g., Reference No. 34] for a review of similar state-of-the-art approaches in the context of 3D microscopy imaging applications, and [scee, e.g., Reference Nos. 5 and 35] for exemplary discussion using deep neural networks to implement reverse models for image reconstruction. FIG. 9 shows exemplary graphs of exemplary results according to an exemplary embodiment of the present disclosure. [see, e.g., Reference No. 5]).

The exemplary RM may include multiple (e.g., three) components (green (procedure 1110), blue (procedure 1120), red (procedure 1130) as shown in FIG. 11). The first component T-1 ( ) is a neural network model that can rigidly align the volume into the canonical reference frame (y→yc, procedure 1110 green in FIG. 11). This component can be trained using supervised learning (alignment loss) [see, e.g., Reference Nos. 36 and 37]. The second component can be a U-Net neural network (procedure 1120, blue in FIG. 11) that can approximate the ground-truth volume by suppressing the noise and increasing the resolution. The third component (procedure 1130, red in FIG. 11) can extend the basic U-Net (blue) into greater depth (red). The resulting deep abstraction hierarchy can be trained to form an abstract map of latent variables that encodes the local anatomy in a probabilistic model.

Exemplary Spatial alignment for co-registration: The first transformation to be learned can be spatial alignment (shown in green in FIG. 11). Exemplary system, method and computer-accessible medium accordingly to an exemplary embodiment of the present disclosure can focus on rigid-body alignment by translation and rotation in the 3D volume. The forward model can contain a random rotation and translation. [see, e.g., Reference No. 36]. Exemplary system, method and computer-accessible medium accordingly to an exemplary embodiment of the present disclosure can be an NN model that can estimate the translation and rotation required to rigidly align the volume to a canonical reference frame. The exemplary system can include both feedforward NNs and recurrent NNs. Recurrent NNs can combine the benefits of a machine learning approach for alignment (rapid, computationally efficient alignment after the computational investment of learning alignment for a specific class of object: human brains) with the benefits of iterative alignment (greater precision at somewhat greater computational cost). The rigid-body alignment (T-1 ( ) green box in FIG. 11) can be trained in a supervised fashion using the ground-truth misalignment (T( ) gray box in FIG. 11) drawn randomly in the FM. Beyond rigid-body alignment, the exemplary system can relate individuals in a common abstract representation. This can facilitate a more general non-rigid alignment that smoothly distorts the volume to align corresponding anatomical structures across different brains. The nonlinear alignment can be implemented with a NN model following recent advances [see, e.g., Reference No. 36]. This exemplary approach builds on the classical volume-based non-rigid alignment methods, but the NN approach may have the potential to find better solutions (by jointly optimizing alignment and abstraction) and is computationally more efficient than classical iterative methods (at the computational cost of having to be learned from data). The non-rigid alignment component can output a canonically aligned volume yc, where each location is associated with a particular part of the brain.

Exemplary Convolutional U-Net for denoising and super-resolution: The exemplary second transformation to be learned can achieve denoising and super-resolution. This transform can be implemented in a U-Net architecture [see, e.g., Reference No. 38]; blue in FIG. 11) that transforms the aligned low-resolution volume yc into an estimate ∧xc of the high-quality ground-truth volume xc (all canonically aligned). The encoder component (yc→z1, z2) can use a deep convolutional architecture that automatically generalizes the learned filters across spatial positions. On the one hand, this can reduce the number of parameters to be learned and can enable data in one position to inform processing in a different position. On the other hand, it can prevent the model from exploiting information about image structure that is specific to particular brain locations. U-Net architectures have previously been successfully applied in different domains [see, e.g., Reference Nos. 39 and 40] including MRI [see, e.g., Reference Nos. 41], to achieve denoising and super-resolution. Once the training architecture is in place to estimate the parameters of the FM and RM, one can optimize over the allowable space of system parameters (e.g., pulse sequence acquisition parameters, reconstruction, and image processing pipelines) in an outer loop to improve the effective system resolution and reliability of the output images ([see, e.g., Reference Nos. 18 and 33].

Exemplary Deep abstraction hierarchy: The U-Net architecture can be extended in depth to represent an abstract latent space of individual human brain anatomy (red in FIG. 11). Latents will span the gamut from a low-level encoding z1 to a high-level encoding zk. In the lower part of the model (which will achieve denoising and super-resolution), the exemplary system used convolutional layers. The abstraction hierarchy can build on these convolutional layers with layers that employ restricted receptive fields, but do not use the weight sharing across locations that defines convolutional layers. Weight sharing can cause the abstraction hierarchy to learn the local anatomy of each part of the human brain separately at higher levels. For example, the brain stem can require different latent features than the occipital cortex. The top layer of the abstraction hierarchy can consist in a set of feature maps zk (each a volume at reduced resolution) that characterize in a compressed format the anatomy of a particular individual brain.

Probabilistic model of human brain anatomy to improve images and inferences. The exemplary component models can be integrated to form a probabilistic model of human brain anatomy and its reflection in T1-weighted MR images. This model will probabilistically capture the variation across healthy individuals reflected in T1-weighted images with millimeter resolution. The deeper understanding of human brain structure implicit to the model can enhance the quality of the images and inferences. Beyond a database of many scans, such a model can eventually enable substantial basic science and applied advances. Exemplary system, method and computer-accessible medium accordingly to an exemplary embodiment of the present disclosure can help human neuroscience understand the natural variability of human brain structure, the relationships between different structural variables, and normal developmental trajectories and how they relate to cognitive development. In terms of applications, the exemplary system can enable earlier and more sensitive detection of deviations from the healthy distribution. In particular, it can enable one to estimate the probability that a given MRI volume is from a healthy brain and to create probabilistic maps indicating exactly where a given brain deviates.

Exemplary system, method and computer-accessible medium accordingly to an exemplary embodiment of the present disclosure can relate to a generative model p(x,y,z)=p(z)·p(x|z)·p(y|x) of healthy human brain anatomy, whose underlying graphical model is z→x→y (FIG. 11). The latent vector z characterizes the anatomy of an individual human brain, and p(z) is the learned prior over the latent space, which characterizes the natural variation across the healthy human population. The brain image x represents the unknown noise-free true image volume (the goal of inference), which reflects the latent individual anatomy z. The brain image y is the output of the new accessible MR system, which in turn reflects the true image x, but is compromised by noise, distortion, subsampling, and misalignment. The FM captures p(y|x), the probabilistic mapping x→y. Conversely, the RM captures p(x|y), the reverse mapping y→x, aiming to recover brain anatomy in a denoised super-resolution image. Finally, p(x|z) is the generative likelihood (i.e., the probability of obtaining the structural brain image x given that the individual's latent representation is z). By improving the generative model for x and y using the large datasets provided by the exemplary accessible MRI system, one can improve (1) the accuracy of the RM and thus the image quality and (2) the quality of the latent space, which will eventually support further inferences for basic science and clinical applications. The components of this probabilistic model can be implemented with deep neural networks (constrained by the physics-based FM). We will leverage all available data to learn the parameters of the model.

Exemplary Model training. Exemplary system, method and computer-accessible medium accordingly to an exemplary embodiment of the present disclosure can combine supervised learning of the forward model p(y|x) and the reverse models p(x|y) with unsupervised learning of the generative models p(x) and p(y). An exemplary approach to training is thus semi-supervised [see, e.g., Reference No. 42]. The training can take place in two phases. In Phase 1, the exemplary system can use a large number of high-resolution T1-weighted MRI volumes acquired at 3T, which can be available in several existing publicly available databases (e.g., the Human Connectome Project, the Alzheimer's Disease Neuroimaging Initiative, the UK Biobank, or the ABCD Study [see, e.g., Reference Nos. 43-46]). Exemplary system, method and computer-accessible medium accordingly to an exemplary embodiment of the present disclosure can use existing datasets and a small amount of data from the exemplary system to set the initial parameters of the FM p(y|x), which is physics-based and so will not require the fitting of many model parameters. Exemplary system, method and computer-accessible medium accordingly to an exemplary embodiment of the present disclosure can then use the high-resolution 3T images as an approximation to the true images x, to provide a basis for the FM to simulate images y of the new accessible MRI system. The resulting training pairs (xi, yi) can be used for supervised learning of the RM p(x|y). In addition, Exemplary system, method and computer-accessible medium accordingly to an exemplary embodiment of the present disclosure can use the existing 3T data for unsupervised learning of p(x), providing a prior to constrain the inference of the true image x. Phase 2 can continue refinement of the probabilistic model using the data flowing in through the cloud as accessible MRI is deployed in many locations. The growing data can constrain the probabilistic model p(y) of the images from the new system, thus continually improving the model's understanding of the imaging system and, one level deeper, of the natural variation of human brain anatomy. Exemplary system, method and computer-accessible medium accordingly to an exemplary embodiment of the present disclosure can use simulated data from remote sites to provide a proof of concept and quantitative evidence demonstrating the incremental learning.

### Exemplary Demonstration in a Heterogeneous Sample of Human Brains

Exemplary system, method and computer-accessible medium accordingly to an exemplary embodiment of the present disclosure can demonstrate and quantitatively validate the new MR system and model-based imaging in 120 human subjects. Exemplary system, method and computer-accessible medium accordingly to an exemplary embodiment of the present disclosure can acquire structural MRI data in each of these participants on the new and conventional systems to evaluate the quality of the images in comparison to existing technology. To engage the challenges associated with imaging and modeling more diverse human brain anatomies than previously attempted, exemplary samples can include a wide age range (preschool-age children and older adults; 4-60 years of age). This age range is motivated by data suggesting that most brain development occurs before 25 years [see, e.g., Reference No. 47] and that most normal aging occurs before 60 years of age [see, e.g., Reference Nos. 48 and 49]. Studies of development and structural aging often rely on adult atlases or study-specific templates. For example, FreeSurfer is the automated software package for processing anatomical images for morphometric analysis. Its longitudinal processing stream generates a within-subject template to increase reliability and statistical power [see, e.g., Reference No. 50], but was developed for young-adult populations and is therefore suboptimal for understanding age-related diversity of anatomy. For example, it assumes that intracranial volume (ICV) is stable in the participant across time, although ICV likely continues to increase up to mid-adolescence [see, e.g., Reference No. 51]. Imaging the human brain across a wide range of ages can provide a formidable challenge for model-based, accessible MRI, and one that aligns with an exemplary object of this disclosure: making MRI accessible globally across diverse and heterogeneous populations. As accessible MRI is deployed throughout the world, it can be possible to learn a probabilistic model of healthy brain anatomy as a function of age and to refine this model automatically and continuously.

Exemplary MRI data acquisition. Exemplary system, method and computer-accessible medium accordingly to an exemplary embodiment of the present disclosure can acquire structural MRI data from 120 healthy individuals on both accessible (exemplary MRI system developed) and standard MRI systems (1.5 and 3T). All participants can undergo 3 MRI scans on a single day: one on the exemplary MR system, one on a 1.5T (GE Artist), and one on a 3T (either Siemens Prisma or GE Signa Premier) system. Participants can be randomly assigned to one of the 3T systems. The participants can undergo a short MR protocol on each system, consisting of T1-MPRAGE and T2-TSE sequences, ensuring whole-brain coverage with a slice thickness of 1 mm.

Subject sample: Participants can be male and female, of any ethno-racial category and ranging in age from childhood (n=40; ages 4 to 12 years), through adolescence (n=40; 13 to 21 years), to adulthood (n=40; 22 to 60 years). There can be 40 subjects in each group because of the heterogeneity of the subject sample. One would expect larger variation across, and even within, the three groups than in a conventional MRI study. 120 subjects can enable one to reliably estimate the image quality of the new system and to perform inferential comparisons treating subject as a random effect, so as to generalize to the population. Exclusion criteria may include: (1) pregnancy; (2) a current or lifetime history of a major medical or neurological problem (e.g., unstable hypertension, seizure disorder, head trauma); (3) presence of a metallic device (ferromagnetic implants or dental braces); (4) current or past history of any psychiatric disorder; (5) active suicidal ideation; (6) IQ<80.

Exemplary Quantification of image quality. It is possible to use the repeated measurements in the 120 subjects to quantitatively assess the quality of the exemplary accessible MRI system. The 3T images from the same subject can serve as a reference for assessing image quality, after co-registration of the brain volumes and scaling of the assessed image to minimize the squared deviation from the 3T reference.

Exemplary Volumetric image-quality assessment: Itis possible to use two measures for assessing the image quality: (1) the peak-signal-to-noise ratio (PSNR) in dB (10·log 10 [max2/MSE], where max is the maximum intensity and MSE is the mean squared error relative to the 3T reference image) and (2) the structural similarity index measure (SSIM, [see, e.g., 57]). In each subject, one can quantify image quality with both measures (a) for the new system, using different image-reconstruction methods, with and without the model-based image enhancement, and (b) for the 1.5 T system. The variation across the 120 subjects can enable one to compute confidence intervals and perform inferential comparisons between the new system and the 1.5T, and among different variants of the new system.

Exemplary Cortical-thickness map quality assessment: In addition to these volumetric image quality measures, it is possible to quantify image quality according to the precision of cortical thickness (CT) maps. As for the MSE and the SSIM, one will use the 3T volume as a reference in each subject. One can use the FreeSurfer software to perform cortex reconstruction, forming polygon meshes along the boundary between gray and white matter and along the pial surface. One can then measure CT at each cortex location as the distance between the two bounding surfaces. One can virtually flatten the cortical sheet for each structural MRI and impose the CT map. Within each subject, one can rigidly (or near-rigidly) align the cortical flatmaps from the new system (for each of its variants) and the 1.5T to the reference 3T cortical flatmap. This within-subject cortical-sheet-based co-registration can rely solely on the folding pattern (curvature map) of the cortex. One will then quantify the quality of the CT map, using the 3T CT map as the reference. To achieve invariance to an additive bias in the CT estimates (which could result from image intensity variation and thresholding for cortex reconstruction), one can use the Pearson correlation coefficient to compare CT maps to the 3T reference maps. As for the PSNR and SSIM, one can compute confidence intervals for the CT-map quality measure and perform inferential comparisons between the new method (different variants) and the 1.5T, and among variants of the new method.

Exemplary Conclusion. To make MR accessible to the world, exemplary system, method and computer-accessible medium accordingly to an exemplary embodiment of the present disclosure can improve the technology by combining hardware, cloud connectivity, and machine learning. Exemplary system, method and computer-accessible medium accordingly to an exemplary embodiment of the present disclosure can include an MR system that can be sited and operated wherever people live through a cloud platform for remote acquisition, archiving, and curation of large data repositories. This can benefit scientific and medical MR, and can be realized by exemplary systems demonstrating the exemplary embodiments according to the present disclosure by accurately imaging brain structure in a highly heterogeneous population.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

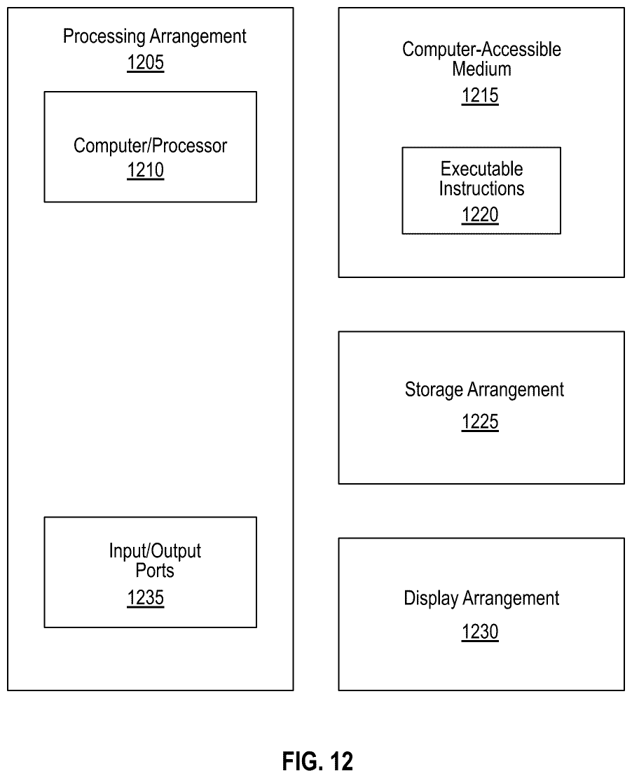

FIG. 12 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 1205. Such processing/computing arrangement 1205 can be, for example entirely or a part of, or include, but not limited to, a computer/processor 1210 that can include, for example one or more microprocessors, and use instructions stored on a computer-accessible medium (e.g., RAM, ROM, hard drive, or other storage device).

As shown in FIG. 12, for example a computer-accessible medium 1215 (e.g., as described herein above, a storage device such as a hard disk, floppy disk, memory stick, CD-ROM, RAM, ROM, etc., or a collection thereof) can be provided (e.g., in communication with the processing arrangement 1205). The computer-accessible medium 1215 can contain executable instructions 1220 thereon. In addition or alternatively, a storage arrangement 1225 can be provided separately from the computer-accessible medium 1215, which can provide the instructions to the processing arrangement 1205 so as to configure the processing arrangement to execute certain exemplary procedures, processes, and methods, as described herein above, for example.

Further, the exemplary processing arrangement 1205 can be provided with or include an input/output ports 1235, which can include, for example a wired network, a wireless network, the internet, an intranet, a data collection probe, a sensor, etc. As shown in FIG. 12, the exemplary processing arrangement 1205 can be in communication with an exemplary display arrangement 1230, which, according to certain exemplary embodiments of the present disclosure, can be a touch-screen configured for inputting information to the processing arrangement in addition to outputting information from the processing arrangement, for example. Further, the exemplary display arrangement 1230 and/or a storage arrangement 1225 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference, in their entireties:
1. Shen F X, Lawrenz F P, Wolf S M. Highly Portable and Cloud-Enabled Neuroimaging Research: Confronting Ethics Challenges in Field Research with New Populations. National Institutes of Health; 2020 August. Report No.: 1RFIMH123698-01.
2. World Health Organization. Global atlas of medical devices. 2017. Available: https://www.who.int/medical_devices/publications/global_atlas_meddev2017/en/3. Geethanath S, Vaughan J T. Accessible magnetic resonance imaging: A review. J Magn Reson Imaging. 2019; 49: e65-e77. doi: 10.1002/jmri.26638
4. Garwood M. Imaging Human Brain Function with Minimal Mobility Restrictions. National Institutes of Health; 2017 September. Report No.: 5U01EB025153-04.
5. Venkate G, Punith Kumara Swamy A, Jambawalikar S, Geethanath S, Vaughan T. REconstruction of M R images acquired in highly inhomogeneous fields using DEep Learning (REMODEL). International Society for Magnetic Resonance in Medicine. 2018. p. 356.
6. Cox S R, Ritchie S J, Fawns-Ritchie C, Tucker-Drob E M, Deary I J. Structural brain imaging correlates of general intelligence in U K Biobank. Intelligence. 2019; 76:101376. doi: 10.1016/j.intell.2019.101376
7. Zeng D Y, Shaikh J, Holmes S, Brunsing R L, Pauly J M, Nishimura D G, et al. Deep residual network for off-resonance artifact correction with application to pediatric body MRA with 3D cones. Magn Reson Med. 2019; 82:1398-1411. doi: 10.1002/mrm.27825
8. Hutchinson M, Raff U. Fast MRI data acquisition using multiple detectors. Magn Reson Med. 1988; 6:87-91. doi: 10.1002/mrm. 1910060110
9. Vaughan J T, Hetherington H P, Otu J O, Pan J W, Pohost G M. High frequency volume coils for clinical NMR imaging and spectroscopy. Magn Reson Med. 1994; 32:206-218. doi: 10.1002/mrm.1910320209
10. Sohn S-M, Vaughan J T, Lagore R L, Garwood M, Idiyatullin D. In vivo M R imaging with simultaneous R F transmission and reception. Magn Reson Med. 2016; 76:1932-1938. doi: 10.1002/mrm.26464
11. Tong G, Geethanath S, Vaughan J T. On the signal strength of Simultaneous Transmission and Reception (STAR) acquisition: EPG simulation and analysis. International Society for Magnetic Resonance in Medicine. 2019.
12. Weigel M. Extended phase graphs: Dephasing, R F pulses, and echoes-pure and simple. J Magn Reson Imaging. 2015; 41:266-295. doi: 10.1002/jmri.24619
13. Feng L, Grimm R, Block K T, Chandarana H, Kim S, Xu J, et al. Golden-angle radial sparse parallel MRI: Combination of compressed sensing, parallel imaging, and golden-angle radial sampling for fast and flexible dynamic volumetric MRI. Magn Reson Med. 2014; 72:707-717. doi: 10.1002/mrm.24980
14. Anand S, Stockmann J P, Wald L L, Witzel T. A low-cost ($500 USD) FPGA-based console capable of real-time control. International Society for Magnetic Resonance in Medicine. 2018.
15. Ravi K S, Potdar S, Poojar P, Reddy A K, Kroboth S, Nielsen J-F, et al. Pulseq-Graphical Programming Interface: Open source visual environment for prototyping pulse sequences and integrated magnetic resonance imaging algorithm development. Magn Reson Imaging. 2018; 52:9-15. doi: 10.1016/j.mri.2018.03.008
16. Layton K J, Kroboth S, Jia F, Littin S, Yu H, Leupold J, et al. Pulseq: A rapid and hardware-independent pulse sequence prototyping framework. Magn Reson Med. 2017; 77:1544 1552. doi: 10.1002/mrm.26235
17. Tong G, Geethanath S, Jimeno M, Qian E, Ravi K S, Vaughan J T Jr. Virtual Scanner. International Society for Magnetic Resonance in Medicine. 2019.
18. Ravi K S, Gecthanath S, Weber J, Vaughan J T. M R Value driven Autonomous MRI using imr-framework. ISMRM Workshop on Machine Learning Part I I. 2018.
19. Jezzard P, Clare S. Sources of distortion in functional MRI data. Hum Brain Mapp. 1999; 8:80-85. doi: 3.0.C O; 2-C″>10.1002/(SICI) 1097-0193 (1999) 8: 2/3<80::AID-HBM2>3.0.C O;2-C
20. Reichenbach J R, Venkatesan R, Yablonskiy D A, Thompson M R, Lai S, Haacke E M. Theory and application of static field inhomogeneity effects in gradient-echo imaging. J Magn Reson Imaging. 1997; 7:266-279. doi: 10.1002/jmri.1880070203
21. Jezzard P. Correction of geometric distortion in fMRI data. Neuroimage. 2012; 62:648-651. doi: 10.1016/j.neuroimage.2011.09.010
22. Cusack R, Brett M, Osswald K. An Evaluation of the Use of Magnetic Field Maps to Undistort Echo-Planar Images. Neuroimage. 2003; 18:127-142. doi: 10.1006/nimg.2002.1281
23. Robson M D, Gore J C, Constable R T. Measurement of the point spread function in MRI using constant time imaging. Magn Reson Med. 1997; 38:733-740. doi: 10.1002/mrm.1910380509
24. Speck O, Stadler J, Zaitsev M. High resolution single-shot EPI at 7T. Magn Reson Mater Phys Biol Med. 2008; 21:73-86. doi: 10.1007/s10334-007-0087-x
25. Juchem C, Nixon T W, McIntyre S, Boer V O, Rothman D L, de Graaf R A. Dynamic multi-coil shimming of the human brain at 7T. J Magn Reson. 2011; 212:280-288. doi: 10.1016/j.jmr.2011.07.005
26. Jimeno M, Geethanath S, Vaughan J T Jr. Open-source Python package for spiral off-resonance correction. International Society for Magnetic Resonance in Medicine. 2020.
27. Sohn S-M, Idiyatullin D, Vaughan J T, Garwood M. System and Method for Simultaneous Radiofrequency Transmission and Reception in Magnetic Resonance Imaging. 2018.
28. Tong G, Geethanath S, Jimeno M, Qian E, Ravi K, Girish N, et al. Virtual Scanner: MRI on a Browser. Journal of Open Source Software. 2019; 4:1637. doi: 10.21105/joss.01637
29. Ravi K S, Gecthanath S, Vaughan J. PyPulseq: A Python Package for MRI Pulse Sequence Design. Journal of Open Source Software. 2019; 4:1725. doi: 10.21105/joss.01725
30. Latourette M T, Siebert J E, Barto R J, Marable K L, Muyepa A, Hammond C A, et al. 30. Magnetic Resonance Imaging Research in Sub-Saharan Africa: Challenges and Satellite-Based Networking Implementation. J Digit Imaging. 2011; 24:729-738. doi: 10.1007/s10278-010-9323-4
31. Ravi K S, Geethanath S. Autonomous Magnetic Resonance Imaging. Magn. Reson Med. 2020 (in press, https://www.sciencedirect.com/science/article/pii/S0730725X20302216).
32. Qian E, Geethanath S, Nielsen J-F, Vaughan J T Jr. Rapid open source prototyping of Magnetic Resonance Fingerprinting using Pulseq to enable multi-site two vendor studies. International Society for Magnetic Resonance in Medicine. 2019.
33. Ravi K S, Geethanath S, Vaughan J T Jr. Self-administered exam using Autonomous Magnetic Resonance Imaging (AMRI). International Society for Magnetic Resonance in Medicine. 2019.
34. Belthangady C, Royer L A. Applications, promises, and pitfalls of deep learning for fluorescence image reconstruction. Nat Methods. 2019; 16:1215-1225. doi: 10.1038/s41592-019-0458-z
35. Parthasarathy N, Batty E, Falcon W, Rutten T, Rajpal M, Chichilnisky E J, et al. Neural Networks for Efficient Bayesian Decoding of Natural Images from Retinal Neurons. Advances in Neural Information Processing Systems 30 (NIPS 2017). 2017.
36. Rohe M-M, Datar M, Heimann T, Sermesant M, Pennec X. SVF-Net: Learning Deformable Image Registration Using Shape Matching. International Conference on Medical Image Computing and Computer-Assisted Intervention. 2017. pp. 266-274. doi: 10.1007/978-3-319-66182-7_31
37. Simonovsky M, Gutiérrez-Becker B, Mateus D, Navab N, Komodakis N. A Deep Metric for Multimodal Registration. International Conference on Medical Image Computing and Computer-Assisted Intervention. 2016. pp. 10-18. doi: 10.1007/978-3-319-46726-9_2
38. Ronneberger O, Fischer P, Brox T. U-Net: Convolutional Networks for Biomedical Image Segmentation. Medical Image Computing and Computer-Assisted Intervention-MICCAI 2015. 2015. pp. 234-241. doi: 10.1007/978-3-319-24574-4_28
39. Park J, Hwang D, Kim K Y, Kang S K, Kim Y K, Lee J S. Computed tomography super-resolution using deep convolutional neural network. Physics in Medicine & Biology. 2018; 63:145011.
40. Liu D, Wen B, Liu X, Wang Z, Huang T S. When Image Denoising Meets High-Level Vision Tasks: A Deep Learning Approach. arXiv. 2017. Available: http://arxiv.org/abs/1706.04284
41. Figini M, Lin H, Ogbole G, D′Arco F, Blumberg S B, Carmichael D W, et al. Image Quality Transfer Enhances Contrast and Resolution of Low-Field Brain MRI in African Paediatric Epilepsy Patients. arXiv. 2020.
42. Wu A, Buchanan E K, Whiteway M, Schartner M, Meijer G, Noel J-P, et al. Deep Graph Pose: a semi-supervised deep graphical model for improved animal pose tracking. bioRxiv. 2020.
43. Van Essen D C, Ugurbil K, Auerbach E, Barch D, Behrens TEJ, Bucholz R, et al. The Human Connectome Project: A data acquisition perspective. Neuroimage. 2012; 62:2222-2231. doi: 10.1016/j.neuroimage.2012.02.018
44. Wyman B T, Harvey D J, Crawford K, Bernstein M A, Carmichael O, Cole P E, et al. Standardization of analysis sets for reporting results from ADNI MRI data. Alzheimers Dement. 2013; 9:332-337. doi: 10.1016/j.jalz.2012.06.004
45. Sudlow C, Gallacher J, Allen N, Beral V, Burton P, Danesh J, et al. U K Biobank: An Open Access Resource for Identifying the Causes of a Wide Range of Complex Diseases of Middle and Old Age. PLOS Med. 2015; 12: e1001779. doi: 10.1371/journal.pmed. 1001779
46. Hagler D J, Hatton S N, Cornejo M D, Makowski C, Fair D A, Dick A S, et al. Image processing and analysis methods for the Adolescent Brain Cognitive Development Study. Neuroimage. 2019; 202:116091. doi: 10.1016/j.neuroimage.2019.116091
47. Gogtay N, Giedd J N, Lusk L, Hayashi K M, Greenstein D, Vaituzis A C, et al. Dynamic mapping of human cortical development during childhood through early adulthood. Proceedings of the National Academy of Sciences. 2004; 101:8174-8179. doi: 10.1073/pnas.0402680101
48. Brickman A M, Khan U A, Provenzano F A, Yeung L-K, Suzuki W, Schroeter H, et al. Enhancing dentate gyrus function with dietary flavanols improves cognition in older adults. Nat Neurosci. 2014; 17:1798-1803. doi: 10.1038/nn.3850
49. Feng X, Guo J, Sigmon H C, Sloan R P, Brickman A M, Provenzano F A, et al. Brain regions vulnerable and resistant to aging without Alzheimer's disease. Ginsberg S D, editor. PLOS One. 2020; 15: e0234255. doi: 10.1371/journal.pone.0234255
50. Reuter M, Schmansky N J, Rosas H D, Fischl B. Within-subject template estimation for unbiased longitudinal image analysis. Neuroimage. 2012; 61:1402-1418. doi: 10.1016/j.neuroimage.2012.02.084
51. Mills K L, Goddings A-L, Herting M M, Meuwese R, Blakemore S-J, Crone E A, et al. Structural brain development between childhood and adulthood: Convergence across four longitudinal samples. Neuroimage. 2016; 141:273-281. doi: 10.1016/j.neuroimage.2016.07.044
52. Kaufman J, Birmaher B, Brent D, Rao U, Flynn C, Moreci P, et al. Schedule for Affective Disorders and Schizophrenia for School-Age Children-Present and Lifetime Version (K-SADS-P L): Initial Reliability and Validity Data. J Am Acad Child Adolesc Psychiatry. 1997; 36:980-988.
53. First M B, Spitzer R L, Gibbon M, Williams JBW. Structured Clinical Interview for DSM-I V-T R Axis I Disorders, Research Version, Non-Patient Edition (SCID-I/N P). New York State Psychiatric Institute; 2002.
54. Wechsler D. Wechsler Abbreviated Scale of Intelligence. San Antonio, T X: The Psychological Corporation; 1999.
55. Oldfield R C. The assessment and analysis of handedness: The Edinburgh inventory. Neuropsychologia. 1971; 9:97-113. doi: 10.1016/0028-3932 (71) 90067-4
56. Hollingshead A B. Four-factor index of social status. 1975.
57. Zhou Wang, Bovik A C, Sheikh H R, Simoncelli E P. Image quality assessment: from error visibility to structural similarity. IEEE Trans Image Process. 2004; 13:600-612. doi: 10.1109/TIP.2003.819861
58. Ravi, Keerthi Sravan, and Sairam Geethanath. “Autonomous magnetic resonance imaging.” Magnetic Resonance Imaging 73 (2020): 177-185.
59. Ravi, Keerthi Sravan et al. “PyPulseq in a web browser: a zero footprint tool for collaborative and vendor-neutral pulse sequence development”. ISMRM 28th Annual Meeting & Exhibition, 2021.
60. Ravi, Keerthi Sravan et al. “Deep learning Assisted Radiological reporT (DART)”. ISMRM 28th Annual Meeting & Exhibition, 2020.
61. Ravi, Keerthi Sravan et al. “Intelligent protocolling for Autonomous MRI”. ISMRM 28th Annual Meeting & Exhibition, 2020.
62. Ravi, Keerthi Sravan et al. “PyPulseq: A Python Package for MRI Pulse Sequence Design”. Journal of Open Source Software, 2019.
63. Ravi, Keerthi Sravan et al. “Self-administered exam using Autonomous Magnetic Resonance Imaging (AMRI)”. ISMRM 27th Annual Meeting & Exhibition. Montreal, Canada, 2019.
64. Ravi, Keerthi Sravan et al. “Autonomous scanning using imr-framework to improve M R accessibility”. ISMRM Workshop on Accessible MRI for the World. New Delhi, India, 2019.
65. Ravi, Keerthi Sravan et al. “Pulseq-Graphical Programming Interface: Open source visual environment for prototyping pulse sequences and integrated magnetic resonance imaging algorithm development”. Magnetic Resonance Imaging, 2018
66. Ravi, Keerthi Sravan et al. “M R Value driven Autonomous MRI using imr-framework”. ISMRM Workshop on Machine Learning, Part I I. Washington D C, USA, 2018.
67. Potdar, Sneha, Keerthi Sravan Ravi et al. “Pulseq-GPI: A single, comprehensive framework for M R method development”. Joint Annual Meeting ISMRM-ESMRB 2018 Annual Meeting & Exhibition. Paris, France, 2018.
68. Ravi, Keerthi Sravan et al. “Implementation of Pulseq in GPI Lab”. ISMRM 25th Annual Meeting & Exhibition. Hawaii, USA, 2017.
69. Gowda et al., “Reconstruction of M R images acquired in highly inhomogeneous fields using Deep Learning (REMODEL)”, ISMRM, Jun. 16-21, 2018.
70. K S Ravi et. al., “PyPulseq in a web browser: a zero footprint tool for collaborative and vendor-neutral pulse sequence development”, MRI 2018 (https://index.mirasmart.com/ISMRM2021/PDFfiles/3760.html).
71. Kelvin J. Layton et al., “Pulseq: A rapid and hardware-independent pulse sequence prototyping framework”, Magnetic Resonance in Medicine, Jun. 7, 2016.

1. A magnetic resonance (“MR”) system for diagnostic imaging, comprising:
a magnet configured to generate a magnetic field to a subject;
a radio frequency (“RF”) analog spectrometer configured to generate RF pulses to the subject;
a scanner configured to detect (i) a resultant field associated with the magnetic field and (ii) resultant pulses associated with the RF pulses; and
an autonomous MRI software application configured to:
a) be activated through a remote mode of operation, and
b) instruct the scanner to remotely detect the resultant field and the resultant pulses.

2. The MR system of claim 1, wherein the magnet is at least one of a superconducting, a solenoid, or a short solenoid with a nonuniform field of less than 5 ppm.

3. The MR system of claim 1, wherein field nonuniformities of the magnet are used for spatial encoding.

4. The MR system of claim 1, wherein a bore of the magnet at least one of improves ergonomics, reduces claustrophobia, or reduces at least one of weight, size or cost of the magnet.

5. The MR system of claim 1, wherein the magnet is made of at least one of HTSC or MgB2.

6. The MR system of claim 1, wherein the magnet has a cooling system, which has at least one of cryo-plate cooling, liquid H2 cooling, or solid N2 cooling, or does not have He2 cooling.

7. The MR system of claim 1, wherein the magnet has at least one of an operating temperature of higher than 4.2K, relaxed manufacturing tolerances, or reduced cryostat.

8. The MR system of claim 1, further comprising at least one of a thermal reservoir to maintain a field for a time period without power or a local generator to energize the magnet.

9. The MR system of claim 1, wherein the MR system is at least one of a housed, operated or shielded in a half-sized or full-sized standard shipping container.

10. The MR system of claim 1, wherein the spectrometer has at least one of an RF transmitter, an RF receiver, a TR switch, a circulator, an isolator, an analog to digital converter (“ADC”), a digital to analog converter (“DAC”), a single transmit signal channel, multiple transmit signal channels, a single receive channel, or multiple receive channels.

11. The MR system of claim 1, wherein the spectrometer is configured to at least one of transmit and receive simultaneously, transmit and receive sequentially, or transmit simultaneously.

12. The MR system of claim 1, wherein the spectrometer is configured to isolate transmit and receive by at least one of time, phase, frequency, space (geometry), or signal magnitude.

13. The MR system of claim 1, wherein the spectrometer is configured to at least one of transmit a magnitude modulated signal, transmit a phase modulated signal, transmit a time modulated signal, transmit a spatially modulated signal, transmit a frequency modulated signal, adjust receiver gain, adjust a receiver frequency and bandwidth, receive a signal modulated in time, receive a phase adjusted signal, or conduct spatial beam steering.

14. The MR system of claim 1, wherein the spectrometer is configured to, using a broad-band receiver or transmitter, at least one of receive multiple nuclear resonance frequencies or excite multiple nuclear resonance frequencies.

15. The MR system of claim 1, wherein the spectrometer is controlled by a field programmable gate array (“FPGA”).

16. The MR system of claim 1, further comprising at least one of a data acquisition unit, a digital user interface, a patient table, a field gradient system, a field shim set, an EMI shield, a magnetic fringe field shield, a secure enclosure, a support suite, or a radiofrequency coil.

17. The MR system of claim 1, wherein the MR system is does not contain any rare earths.

18. The MR system of claim 1, wherein the MR system is configured to be networked with other MR systems using a cloud network.

19. The MR system of claim 1, wherein the MR system is networked using wireless or wired networking protocols.

20. The MR system of claim 18, wherein the other MR systems include scanners which are synchronized.

21. The MR system of claim 11, wherein the scanners are configured to communicate using the cloud network to exchange at least one of protocols, data or predictive analysis.

22. The MR system of claim 1, wherein the MR system is at least one of operationally sustainable, reliable, or deliverable using a transportation vehicle.

23. The MR system of claim 1, wherein the mode of operation is at least one of a voice command, a visual user-interface command, a QR code, a smart device.

24. The MR system of claim 1, wherein the mode of operation does not require human input to at least one of acquire, reconstruct, assess or report data.

25. The MR system of claim 1, wherein:
the autonomous MRI software application has an optimization image acquisition module for higher MR values; and
the higher MR values are diagnostic information per unit cost or unit time.

26. The MR system of claim 1, wherein the optimization image acquisition module interacts with a scanner on a cloud.

27. The MR system of claim 1, wherein the autonomous MRI software application is configured to at least one of (i) determine acquisition parameters through at least one of integration of MR physics, AI search strategies, patient derived statistics, or electronic health records, or (ii) denoise MR data to accelerate acquisitions using at least one of native or learned noise structures.

28. (canceled)

29. The MR system of claim 1, wherein the autonomous MRI software application is configured to at least one of (i) exploit transfer learning to leverage native noise denoising, or (ii) integrate at least one of cognizance, reflectivity, adaptivity or ethical compliance rules to transform the MR system into an intelligent system.

30. (canceled)

31. The MR system of claim 1, wherein the autonomous MRI software application is configured to incorporate cognizance through intelligent slice planning.

32. The MR system of claim 1, wherein the autonomous MRI software application is configured to at least one of:
(i) incorporate at least one of cognizance through intelligent slice planning, reflectivity through intelligent protocolling, adaptivity through user intervention for MR exams, taskability through voice interaction, or ethical behavior through patient information encryption in speech to text or text to speech transformations,
(ii) optimize for increased value a ratio of diagnostic information to a cost, wherein at least one of:
the diagnostic information is related to qualitative MR contrasts or quantitative tissue parametric maps; and
the cost is associated with a time spent in the scanner or scanning fees
(iii) enable a remote operation through at least one of self-scanning, monitoring, performing consistency checks, flagging degradation or escalating potential failure modes.

33. (canceled)

34. (canceled)

35. The MR system of claim 1, wherein at least one of the self-scanning is accomplished by the interplay between a user-node, a cloud and the scanner with a user-node controlling the other two components; or the monitoring of the scanner is performed by the use of acquisition associated with a pattern recognition technique.

36. The MR system of claim 1, wherein the autonomous MRI software application is configured to (i) utilize pattern recognition outputs of an acquisition to classify patterns associated with a system status and a degradation status, (ii) flag at least one of system degradation of hardware and networking components including at least one of the magnet, a gradient, or a cloud connectivity, or (iii) control a console comprising a field programmable gate array (“FPGA”) device.

37. (canceled)

38. (canceled)

39. The MR system of claim 38, wherein the FPGA device is configured to at least one of (i) adhere to standards for pulse sequence programming, or (ii) interface with an image guided radiation therapy platform.

40. The MR system of claim 38, wherein the FPGA device includes a large range of transmit and receive channels.

41. The MR system of claim 40, wherein the FPGA device is configured to operate at high sampling rates to accommodate high speed streaming of data experienced in simultaneous transmit and receive acquisitions.

42. The MR system of claim 41, wherein the FPGA device is configured to provide real-time feedback to a user-node to correct for artifacts including patient motion or load changes.

43. (canceled)

44. The MR system of claim 1, wherein the scanner is configured to at least one of acquire images in inhomogeneous fields; integrate electromagnetic simulation and pattern recognition-based acquisition; capture image in highly non-uniform magnetic fields to account for a short bore length; utilize pattern recognition methods including at least one of fingerprinting, frequency swept pulses, or selective excitation; or encode one whole image in a single echo with multiple receiver coils.

45. The MR system of claim 1, wherein the single echo at least one of achieves acquisition times of an order of the echo times of the desired contrast; reduces radio-frequency power deposited in a patient compared to gold-standard spin and gradient echo sequences; reduces peripheral nerve stimulation in patients compared to gold-standard spin and gradient echo sequences; reduces gradient noise compared to gold-standard spin and gradient echo sequences.

46. The MR system of claim 1, wherein the scanner is configured to at least one of generate common contrasts including T1 weighted, T2 weighted, or diffusion weighted imaging, using conventional and simultaneous transmit and receive methods; utilize pattern recognition acquisition-reconstruction methods to produce quantitative tissue parametric maps to simultaneously generate qualitative and quantitative MR data; utilize a vendor-neutral, open source library for development to aid rapid prototyping and development; generate acquisitions in a web-browser to enable cloud generation of acquisition files; utilize pattern recognition methods to generate tissue specific magnetization evolutions to provide quantitative imaging parameters including T1-map, T2-map, or apparent diffusion coefficient map; gauge and detect system degradation including the deterioration of the coils, or console, using pattern recognition methods; estimate temperature using new pulse sequences to provide safety checks above and beyond specific absorption rate methods.

47. The MR system of claim 1, wherein the scanner is configured to at least one of acquire images in inhomogeneous fields using deep learning; exploit system priors including B0 or B1 fields, or subject priors including anthropomorphic details, or cardiac motion pattern, to integrate intelligence in image reconstruction.

48. The MR system of claim 47, wherein the deep learning is configured to at least one of obtain accurate and robust reconstruction in the presence of noise and motion, speed up acquisition or provide repeatable quantitative imaging measures.

49. The MR system of claim 47, wherein the deep learning is configured to at least one of reconstruct data from Cartesian and non-Cartesian trajectories to accelerate image reconstruction computation and reduce artifacts due to aliasing or gridding; translate pattern recognition derived acquisitions to compute quantitative maps in an accelerated manner; or utilize cloud or local computing to perform reconstruction methods related to Cartesian or non-Cartesian data.

50. The MR system of claim 49, wherein the reconstruction methods include at least one of conforming to global (file) standards on acquisition, reconstruction, image analysis or communication (DICOM); or transformation of raw data to clinically valuable and interpretable quantitative parametric maps or statistics.

51. The MR system of claim 49, wherein the maps facilitate clinical assessment or enable inclusion of Electronic Health Record obtained and MR data to predict trends and outcomes.

52. The MR system of claim 49, wherein the reconstruction methods are configured to estimate quantitative MR parameters jointly through randomization of acquisition parameters, estimating gradient warp and non-linearities through calibration and deep learning, or motion estimation through signal analysis from the gradient and radiofrequency coils.

53. The MR system of claim 1, wherein the autonomous MRI software application includes a quality assurance module configured to at least one of guarantee standardization of image quality by flagging presence of artifacts including wrap around, Gibbs ringing, or motion artifacts, during scan time to enable rescans; check for consistent scanner operation, consistent coil performance, anatomy coverage, or missing acquisitions in protocol to provide a baseline image quality for downstream analysis; calculate image quality metrics including reference and non-reference methods to track image quality over time to detect any potential scanner degradation; or identify, recognize and report system degradation based on predetermined responses to random configurations of test signals on each of the hardware components.

54. The MR system of claim 53, wherein the quality assurance module is configured to include random gradient waveforms to test pre-determined point spread functions of such a k-space trajectory.

55. The MR system of claim 1, wherein the scanner is configured to run multiple diagnostic applications related to different anatomies and pathologies.

56. The MR system of claim 1, wherein the autonomous MRI software application is configured to at least one of translate MR data and images into clinically meaningful metrics to characterize structure, function or metabolism of an anatomy of interest; utilize deep learning to calibrate quantitative imaging outcomes per subject and per population; generate a subject-readable report using deep learning that combines subject information, imaging data or radiologist's expertise; provide a digital health record that evolves over time to record a transition of health to disease and potential reversal; or be accessed via an application store on a smart device by users in a configurable manner.

57. A magnetic resonance (“MR”) method for diagnostic imaging, comprising:
generating a magnetic field to be directed to a subject using a magnet;
generating radio frequency (“RF”) pulses to the subject using an RF analog spectrometer;
detecting, with a scanner, (i) a resultant field associated with the magnetic field and (ii) resultant pulses associated with the RF pulses;
activating an autonomous MRI software application through a remote mode of operation, and
using the autonomous MRI software application, instructing the scanner to remotely detect the resultant field and the resultant pulses.

58. A computer-accessible medium having magnetic resonance (“MR”) software for diagnostic imaging thereof, wherein, when instructed, the MR software configures a computer processor to execute procedures comprising:
generating a magnetic field to be directed to a subject by controlling a magnet;
generating radio frequency (“RF”) pulses to the subject by controlling an RF analog spectrometer;
detecting, by controlling a scanner, (i) a resultant field associated with the magnetic field and (ii) resultant pulses associated with the RF pulses;
activating an autonomous MRI software application through a remote mode of operation, and
causing the autonomous MRI software application to instruct the scanner to remotely detect the resultant field and the resultant pulses.
